SEQUENCE LISTING (25 sequences)

SEQ ID NO: 1 - DNA, 2007 bp, Homo sapiens
aaaggtgggagttctttccgggataattttgacaagaggagctgtcattatgaacatggtgggtatgagcgcccgccttcacactgccaggagaatgatggaagcgtggagatgagggatgtccacaaggaccaacaactaagacacactccttatagcatccgatgcgaaagaagaatgaaatggcatagtgaagacgaaatccgtattaccacgtggagaaatagaaaacctccggagagaaaaatgagtcagaacacacaggatggatacacaaggaactggtttaaggtcacaattccttacgggataaagtatgacaaggcatggctaatgaattcaatccagagccattgcagtgaccgcttcactccggttgatttccactacgtccgaaatcgggcatgcttctttgtccaggatgctagcgctgcctccgcattgaaggatgtcagttataagatttatgatgatgagaaccaaaagatatgtatatttgtcaatcattctactgcgccctactctgtgaagaataagttgaagccaggccaaatggagatgctaaagctgaccatgaacaaacggtacaatgtctcccagcaagctcttgatctccagaatctccgctttgacccagacttgatgggccgtgacattgatataatcctgaatcgaagaaactgcatggctgccaccctgaagatcattgaaagaaatttccctgagctgttgtctttgaacttgtgcaacaacaagctgtaccagctggatggcctttctgacattacagagaaggctcccaaagtcaagaccctgaatctctccaaaaataagctggagtcggcgtgggagttgggcaaggtgaaagggctgaagctcgaagagctatggctagaagggaacccgttgtgcagcaccttctcggaccagtccgcctatgtaagtgccatccgggattgtttccccaagttgttacgcctggacggccgagagttatccgcaccagtgattgttgacattgacagctctgagacaatgaaaccctgcaaggaaaactttactggatctgagaccctaaagcatttagtcctgcaattcctgcagcagtattactcgatctatgactctggagatcgacagggtctcctcggtgcttaccacgatgaggcctgcttctccttggctattcccttcgaccccaaggactcagccccgagcagcttgtgcaagtactttgaggatagcaggaatatgaaaacactcaaggacccctacctgaagggggaactgctgaggcgcacaaaacgtgacattgtggactccctcagtgcgttgcccaaaactcagcatgacctcagctccatcctggtggacgtgtggtgccagacggaaaggatgctctgcttttctgtcaatggggttttcaaggaagtggaaggacagtctcagggttctgttctcgccttcacccggaccttcattgctacccctggcagcagttccagtctgtgcatcgtgaatgacgagctgtttgtgagggatgccagcccccaagagactcagagtgccttctccatcccagtgtccacactctcctccagctctgagccctccctctcccaggagcagcaggaaatggtgcaggctttctctgcccagtctgggatgaaactggagtggtctcagaagtgccttcaggacaatgagtggaactacactagagctggccaggccttcactatgctccagaccgagggcaagatccccgcggaggccttcaagcaaatctcctaaaaggagccctccgatgtcttctttgtcttcgttcacatcctctttgtttcctcttttcaccagcctaaggcctggctgaccaggaagccaacgttaacttgcaggccacgtgacataaccacccaaagagccagttgctctgtgtattcgccccactcatgatcaccattttattttcataataaagagtgacgttacacgttaaaaaaaaaa

SEQ ID NO: 2 - DNA, 1576 bp, Homo sapiens
  misc_feature (810)..(1025): Splice region
cgggcgttcgcctctacgttccgcgcgggagcccacgcgcggttcgtccggaacccacagaccagagacgcaggtcccagccttttcggtgtcggcgccagttcccggaggagcggacatgagtgaaagccaggatgaagttcctgatgaagttgagaaccagtttatattgcgtctgcctctggaacatgcttgtactgtcaggaacctagcacgttctcaaagtgtcaagatgaaggataaactaaaaattgacttattgcctgatgggcgccatgcagttgttgaagtagaagatgtcccactagctgctaagctggttgacttgccttgtgttattgaaagcctgagaacgcttgataaaaaaaccttttataaaacagcagacatttctcagatgcttgtgtgcactgctgatggtgatatccacctttctccagaagaaccagctgcctctactgatcctaatatagtcaggaaaaaagaaagggggagagaagaaaaatgtgtctggaagcatggcattacgccaccacttaagaatgtcagaaagaaaaggttccggaaaacacaaaaaaaggtccctgatgtcaaagaaatggaaaaaagcagctttactgagtacattgaatctccagacgtggaaaatgaagtaaagagactgctgcgttcggatgctgaagccgtaagtacccgttgggaagtcattgctgaagatggaaccaaggaaatagaaagtcaaggctccatcccaggatttttgatatcctcgggaatgagcagccacaagcagggtcatacctcgtcagaatatgatatgcttcgggagatgttcagtgattctagaagtaacaatgatgatgatgaggatgaggatgatgaagatgaggatgaggatgaggatgaagatgaagacaaagaagaggaggaggaagattgttctgaagagtatctggaaaggcagctgcaggccgagtttattgaatctggccagtatagggcaaatgaaggtaccagttcaatagtcatggaaattcagaagcagattgagaaaaaggagaaaaagctccataagattcagaataaagcacaaagacagaaggatctcatcatgaaagtggaaaacctgacactcaagaatcattttcagtctgtgctggagcagcttgagttacaggaaaaacaaaaaaatgagaagctcatttccctacaggaacagttgcagcgttttctgaagaagtgaggagagccattggcgtggcccacaaaccttggattcaccatccagactgcagattggatgaaactgtgcacgtttttgtcccttcagttgccttatgttgaatcagttatatttatctgtaccttctttgctacttaaaatatgctcacagtttgtagtcatgtagaaaaggccagtataaaaatgtagtagacttaaagaccccactcgaccatgagactttctctttgtcaatttgggaattattattattaatttagaaatggggtcttgttatgttgcccaggctgaattcagactcctgggcttaagggatccttccgcctcagcctc

SEQ ID NO: 3 - DNA, 833 bp, Homo sapiens
ggccacccgcctttcactatccgccattcttgtcacctcagctgctgccctcgctaccgcaccgacttcgcccgtgtgctcgcctgcacttgcgctgcccgccatggccaccgcccagccgtcgcaggtgcgccagaagtacgacaccaactgcgacgccgccatcaacagccacatcacgctggagctctacacctcctacctgtacctgtctatggccttctacttcaaccgggacgacgtggccctggagaacttcttccgctacttcctgcgcctgtcggacgacaaaatggagcatgcccagaagctgatgaggctgcagaacctgcgcggtggccacatctgccttcacgatatcaggaagccagagtgccaaggctgggagagcgggctcgtggccatggagtccgccttccacctggagaagaacgtcaaccagagcctgctggatctgtaccagctggccgtggagaagggcgacccccagctgtgccacttcctggagagccactacctgcacgagcaagtcaagaccatcaaagagctgggtggctacgtgagcaacctgcgcaagatttgttccccggaagccggcctggctgagtacctgttcgacaagctcaccctgggcggccgcgtcaaagagacttgagcccagatgggccccacagccacggggtcccttccctgggtcaggccactaggcggggcgtgcatgttgccctttcagaacgttctcttcagttttatctttcagttttaccattgttagcaaaaaagttatctggttctcaaagcaataaaggtgtccataaaaaaaaaaaaaaaaa

SEQ ID NO: 4 - DNA, 8370 bp, Homo sapiens
atgcccagtgatgccaaagacagtgttaatggtgaccttttgttaaattggacaagtcttaaaaatattttaagtggtcttaatgcttcttttcctcttcacaacaatactggctcaagcacagtcactacttcaaaatccatcaaagacccaagactgatgaggagagaagaaagtatgggagaacagagtagtactgcaggcttaaatgaggttttgcaatttgagaagagttcagataatgttaattcagaaataaaatcgacaccatctaattctgcctcctcctcagaagttgtccctggtgatcgtgctgttcttactaatggtttggataccccttgctttaaaacttctgttaatgattcacaatcttgggctcacaacatgggctctgaggactatgactgtatacctcccaataaagttaccatggcagggcaatgtaaggaccaaggtaatttttccttcccaatttctgtgtcaaatgtagtgtcagaggttgagaaccaaaaccacagtgaggagaaggctcagagagcccaacaggagtccggtaatgcttatacaaaagagtacagtagtcacatttttcaggactcgcagtcttctgatttaaaaacaatttatcagactggttgccaaacgtctacagtttttccactcaaaaagaaagtaagcattgatgaataccttcaaaatactggaaagatgaaaaacttcgctgacctggaagacagttccaaacatgaagaaaagcaaacttcatggaaagaaattgataatgatttcactaatgaaacaaaaatcagtccaatagataattacattgttttgcaccaagaatacaaagagagtgagagtcataattcttttgggaaaagctgtgataaaatattaattactcaagagttagaaataacaaaatcttctacatctaccataaaggataaggatgaactagatcatctagcattggaatggcaaattactccaagttttgagagcctgtcacaaaagcatcctcagcactctgtggagtatgagggtaacattcatacaagtttagccattgctcaaaagctaatggaactgaaattggggaaaataaatcaaaattatgctagcattataactgaagctttcccgaaaccaaaagacataccccaggccaaagaaatgttcattgatacagttatttcatcttataacatagaaacagctcatgacagttcaaattgcagcataactagagaacatatatgtgtccataggaaaaatgaaaatgaaccagtgtcattagagaacattcagagagactataaagaaactgcttatgttgaagataggggtcaggatcacaatctgttctgtaattcacagttaagcaatgatatatggctgaatgttaatttcaaaaaacaaacagatagagaaaaccaaaatgaggctaaagagaatagtgcttcatgtgtagaaaacaacatagagaacatatatggagacaaaaagcaggattctcatacaaacgaaaatttcagcaatatagatgaaaaggaggacaaaaattaccacaatatagaaattttgagttctgaagaattttctactaaatttaacttgatttgcagagaagataatgcagtgtcagcagcaactgcattattagagagtgaagaagataccattagtgccgtgaaacaaaaagatactgaaaatactggaagaagtgtagagcatttggcttccacgacatttcccaaaactgcaagttcttcagtgtgtgtagcctcaaatgctgcaatacagatagctagtgctactatgcctgcattaagcctaaataatgacgatcaccagatataccagtttaaagaaacttgttcttctgaaagtccagattttggtttgttagtaaaacatagggtttctgattgtgaaattgatacggataaaaataaatcacaagaatcatttcatcaatcaataaatgagaacttagttcttcagagcattgaattggaaagtgaaattgaaatagaattagaagattgtgatgatgcttttatatttcaacaagatacacatagccatgaaaacatgctttgtgaagaatttgtgacctcatataaggctctgaagtctcgtatcagttgggaaggtctgttagcacttgataacggggagatggaagttttggaaagcaccacaggaagggagaatagtgatcagcattattctaaggaaagtaactatttttattcctctacacaaaacaatgaaacagaacttaccagcccaattttacttccagatctacaaattaaaattactaatatatttaggccaggattcagcccgacagctgactcccttgcattgaaagatagtttttgcacacatgtaactgaagccacaaaaccggaaataaataaggaagatggagaaattctaggatttgacatttattcccagccttttggtgaaaatgcagattatccatgtgaagataaagttgataatataaggcaagaatcagggccagtgagtaactctgaaatctccctttcttttgacttgagtcgtaatacagatgtgaatcatacgtctgaaaatcagaacagtgaatctttgtttactgaaccttctaatgtcacaacaatagatgatggaagcagatgtttctttacaaaatcaaaaactgactataatgataccaaaaataaaaaggaggtagaatcaagaattagcaaaaggaagctacatatatcttccagggatcagaacataccacataaagatttaagacgacataaaatttatgggagaaagaggaggctaaccagtcaagactcatctgagtgtttctcttcattatcccaaggacgaattaaaacattttcacagtcagaaaagcacattaagagtgtcctaaatatcctaagtgatgaagcatctttatgtaaaagcaaatgtctttccagaaaactagacaaagcagttgttcacttaaaaaaagctcatagaagagttcacacatctttgcagcttataactaaagtaggagaagaaagaaagggcccattaccaaaatcatatgcagtaatatgcaataatttctgggaaagttgtgaccttcaaggttatagttctgtgtctcaaagaaaatattattctactaagcatttttcgtcaaaaagaaaatatgacaaacggagaaagaaaagagctccaaaagctgatatttctaaatcattaacccatgtgtcaaagcacaagtcttataaaacaagtggagagaaaaaatgcctttctaggaaaagtatggctagcagtgtctcaaaaagtcaccccaccaccagtcacatgggagaattttgtaatcaagaacatcctgaatcacagttgcctgtatcctccacatcccaaagtacaagtcagtcagtttattataatagcagtgtaagcaatccaagtttatcagaagaacatcagcccttttctggaaaaactgcatatctgttttccccagaccactcagatgagaaactaatagaaaaagaaaatcaaattgatacagcatttttatctagcactagtaaatatgaaaagcttgaaaaacattcagcaaatcataatgttaaagatgcaactaaagaaaacagttgtgacgctaatgaagtaataaatgaaagtaattctgtatctttaagttgcataaaagaaaacataaattctagtacaggcaacgattgtgatgcaacttgcataggtcacacaaaggcgaaaactgacgtacttatatcagtcttagattcaaatgtgaagcactttttaaatgatctctaccaacaaggtaaccttattttatctgattgtaaaagaaacctggaagtaaagtggacagatcctattgagagacccaaacaaagcattattacaggaaacttccttatgggcccattaaacctaactttgatagcaagtaaaaagtacagtattcctcaggtatcagccgctgcagtgacagatagtgagggagaatcttcaaaatcttacttggataagcagagaattcttactgtagattcttttgcagcatccagtactgtaccacactgtgagcagagctgtagagaaaaagagcttctaaagacagaacagtgctcttcaggtaattgcctccatacagatgggaatgaaacaaatgtcactgagaattatgagttggatgtagcatcaggaactgaagaagataaaagttatggggaaaatatagtggaattatcttccagtgatagttctctgcttttaagagataatgtaaaaggctcctcttcagaaacatgtattgtgaagaaagacactgaggacagaataacgtggaaagttaaacaagcggaaaaagcaaaagattctgtttacaaaagaagcatgactgaaggatcaactgttaatactgagtacaaaaatcaaaagaatcagatctcagaagaatcctgcttaaatgagaaaattattacaactaacttgattgattcccatctgagcactaaaaatactaccactgagtcagtccctttgaagaacacagtttctaatccgcttaacaaaagagagaagaagggggaaattaaagttagtaaagactcgcagtctgacttgacattacattcagaaatagcctatatttccaaaccaggaattctaggagttaatcatacgcctattttacctgcccactctgaaacctgtaaagtccctactcttctgaagaaacctgcgtcatacgtgagtgattttaaagaaaaacattgctcagctaatcatacggcccttatagctaatctatctcaaattttgcagagggcagatgaagcatcatctttgcagattctacaggaagaaactaaggtttgtctaaatattctccctttatttgtggaagcttttgaaagaaagcaagaatgttcagttgaacaaatcctgatttcaagagaactgttggtagaccaaaacctgtggaataattgcaaacacacattaaaaccatgtgctgttgacactttggtagaacttcaaatgatgatggaaacaattcaattcattgaaaacaaaaaaaggcacttagaaggtgaaccaacattgcgaagcttgctttggtatgatgaaacactgtatgctgagcttcttggaaaaccacgtggatttcaacagcagtctaatttctatcctggtttccaaggaagattaaaatataatgcattctgtgagttacagacttaccatgatcaattagttgaattgcttgaagaaacaaaaagggaaaagaattcatactatgtattcttaaagtacaaacgacaggttaatgaatgtgaagccataatggagcattgttccgattgctttgatttttctctttctgttccatttacctgtggagttaactttggagatagtttagaagacctggaaatcttaagaaaaagtactttaaagttgatcaatgtatgtggggactctcctaaagttcattcgtatccaggaaaacaggaccatctgtggattatcatagaaatgatctcctcaaaggttaattttattaagaacaacgaggcagtacgtgttaaaatatctctttatggtctggaacatatcttttttgatgctgcaaaaaatcttgtttggaaagagagaacacaatccttcagcaaaaaatactcacaaaagaaggacgaagaaaggctactcagagtgaataaatgtgccttttctaagttgcagaagatatatgatactttgtctaaagatttaaacaatgaaccaatttcccctattgggcttgaggaggatactataattgcttccagaaagtcagatcatccaataaacgaagcaacaattagcatagaaaattctaaatttaacagtaatttgcttgcacacccagatatttgttgtattagtgagatattggatcaggctgaatttgcagaccttaaaaaattacaggatctcaccttgagatgtacagatcacttagaaattttaaaaaaatactttcagatgctacaagataataacatggataatatttttatcacagaagaaaatgttttagacgtggtgataaaccacagccatgaggctatcattttaaagcctgaagctattgaaatgtatattgaaatcgtcatggtctcagaaacaattcactttcttaaaaactcaatagcaaagaaactagacaaacagaggtttcgaggtatgctttggtttgatttgtcacttcttcctgagctggttcagtgccaagaaaaaatggcttctttttcatttcttaaagataactcaacagatgtttgcctttggaaagtgatagagactgctgtttccgaacttaagaaagatctggatattatctgcaaatataatgaagctgttaattgctcatatgctattcatttgctctcaagagaacttcaagaactttcagaaataaaaaagcttctgaagaagtccaagtattttatttccacatatattgactttgtgccatatatagcatccataaattatggaagcactgtgacagagttagaatacaactacaatcaattttctacactgctgaagaatgtaatgtctgcccctaggaaagatttaggaaaaatggcccacattaggaaagtcatgaaaacgattgaacatatgaagatgatatgtactaaaaatgctgaactaaccatttcctttttcctatgccaaatgctgtataacagaaggaagattttacagctgaagagaaaagaaaaaatgaatattcatattgtaaaacctggggaaaataacaataaatttagtatttctacgatgttgcccccagtatcagagtgcataaacaaaaacatctcaaattcctctaaaaaacgaccgagcactgtagacaaatgtgaagactctcaggaacaacagcaagatactactgtttccagttgtaaaaagctaaaggtagacatgaaagatgtcacaaaaatcaacagagaaaaggcaacattcaagcatccaaggactacaggatctcatcccaaaagcgaaaacaaaatagtaccaagttcatgtgacagtctgaaaagaaatcatttaacgccaaaaaaggttgaaatgcaaagatcactacctggctcacttttacccttagagaacccaaaagacacttgcgcatcaaagtcggaaagcaaaatagacttaactgtttcatctgatcacttcagtggacaacaggaaaatttaaatagcatgaagaaaagaaatgtgaacttcagtgctgctgaaacaaaaagtgataagaaagattgtgctgcttttgcaatttgtgaccaaaaaagtgtacatggcacattttcaccagaccatgggacgcttttgcagaaatttcttaaaaattccccagatcccacccaaaaatcctgcctttctgatataaacccagaaactgatgtttctcttgtgcctgatgcgtcggtgctctcaaagccaattttctgttttgtgaaagatgtccatcctgatctagaaatgaatgacacagtctttgaacttcaagataatgatatagtaaattcatctattaaaaattcctcatgcatgacttctccagaacccatctgtatccagaacaaaattcctactctgcagataaacaaactacagcctacagaaactgagtcagaggacaaatacatgaaggatacattgaatcccaatactgtgcatacttttggagcatctgggcatataacccttaatgtgaatcaaggagcagagtactctctttctgaacaacagaatgacaaaaattcaaaagtcctaatgcagaatgctgccacatattggaatgaacttccacagtctgcatgtaacccaacatataattcttctgagcatttatttggaacttcatatccatactctgcttggtgtgtttatcagtacagcaacagcaatggcaatgccattacccagacataccaagggataacatcatatgaagtacagccatctccttctgggctgttgaccacagttgcaagtactgcccagggcacacattctaatcttctgtactctcaatattttacttattttgcgggggagccacaagcaaatggctttgtgccagtgaatgggtattttcaatctcaaatacctgcttctaattttcggcagccaattttttcacaatatgcttctcatcagccattaccacaagctacatacccttaccttcctaatcgatttgtgcctccagaagttccttgggtttatgctccatggcaccaagaatcctttcatccaggacactga

SEQ ID NO: 5 - DNA, 2969 bp, Homo sapiens
ttagctgaattccggcagggggactgcaaaggccaatgtgcatgaaaaagactatattcctgttaagggggaagtttgtattgccaagtacactgttgatcagacctggaacagagcaatcatacaaaacgttgatgtgcagcaaaagaaggcacatgtcttatatattgattatggaaatgaagaaataattccattaaacagaatttaccacctcaacaggaacattgacttgtttcctccttgtgccataaagtgctttgtagccaatgttatcccagcagaagggaattggagcagtgattgtatcaaagctactaaaccactgttaatggagcagtactgctccataaagattgtcgacatcttggaagaggaagtggttacctttgctgtagaagttgagctgccaaattcaggaaaacttttagaccatgtgcttatagaaatgggatatggcttgaaacccagtggacaagattctaagaaggaaaatgcagatcaaagtgatcctgaagatgttggaaaaatgacaactgaaaacaacattgtcgtagacaaaagtgacctaatcccaaaagtgttaactttgaatgtaggtgatgagttttgtggtgtggttgcccacattcaaacaccagaagacttcttttgtcaacaactgcaaagtggccgaaagcttgctgaacttcaggcatcccttagcaagtactgtgatcagttgcctccacgctctgatttttatccagccattggtgatatatgttgtgctcagttctcagaggatgatcagtggtaccgtgcctctgttttggcttacgcttctgaagaatctgtactggtcggatatgtagattatggaaactttgaaatccttagtttgatgagactttgtcccataatcccaaagttgttggaattgccaatgcaagctataaagtgtgtactagcaggagtaaagccatcattaggaatttggactccagaagctatttgtctcatgaaaaaacttgtacagaacaaaataatcacagtgaaagtggtggacaagttggaaaacagttccctggtggagcttattgataaatccgagacgcctcatgtcagtgttagcaaagttctcctagatgcaggctttgctgtgggagaacagagtatggtgacagataaacccagtgacgtgaaagaaaccagtgttcccttgggtgtggaaggaaaagtaaatccattggagtggacatgggttgaacttggtgttgaccaaacagtagatgttgtggtctgtgtgatatatagtcctggagaattttattgccatgtgcttaaagaggatgctttaaagaaactcaatgatttgaacaagtcattagcagaacactgccagcagaagttacctaatggtttcaaggcagagataggacaaccttgttgtgctttttttgcaggtgatggtagttggtatcgtgctttagtcaaggaaatcttaccaaatggacatgttaaagtacattttgtggattatggaaacatcgaagaagttactgcagatgaactccgaatgatatcatcaacatttttaaaccttccctttcagggaatacggtgccagttagcagatatacagtctagaaacaaacattggtctgaagaagccataacaagattccagatgtgtgttgctgggataaaattgcaagccagagtggttgaagtcactgaaaatgggataggagttgaactcaccgatctctccacttgttatcccagaataattagtgatgttctgattgatgaacatctggttttaaaatctgcttcaccacataaagacttaccaaatgacagacttgttaataaacatgagcttcaagttcatgtacagggacttcaagctacctcttcagctgagcaatggaagacgatagaattgccagtggataaaactatacaagcaaatgtattagaaatcataagcccaaacttgttttatgctctaccaaaagggatgccagaaaatcaggaaaagctgtgcatgttgacagctgaattattagaatactgcaatgctccgaaaagtcgaccaccctatagaccaagaattggagacgcatgctgtgccaaatacacaagtgatgatttttggtatcgtgcagttgttctggggacatcagacactgatgtggaagtgctctatgcagactatggaaacattgaaaccctgcctctttgcagagtgcaaccaatcacctctagccacctggcgcttcctttccaaattattagatgttcacttgaaggattaatggaattgaatggaagctcttctcaattaataataatgctattaaaaaatttcatgttgaatcagaatgtaatgctttctgtgaaaggaattacaaagaatgtccatacagtgtcagttgagaaatgttctgagaatgggactgtcgatgtagctgataagctagtgacatttggtctggcaaaaaacatcacacctcaaaggcagagtgctttaaatacagaaaagatgtataggacgaattgctgctgcacagagttacagaaacaagttgaaaaacatgaacatattcttctcttcctcttaaacaattcaaccaatcaaaataaatttattgaaatgaaaaaactggtaaaaagttaagtaagttaaatcgtatgttttcgcctcttctgtgatcaccaataggacatcttcaggcatattggcaggatagagctaatggagtgaaacctattgtaaggctgtactttcgtgatttaatgacctgaggtttggtcataatgcttctgctgtttttgtaggtttatctgatcgttttcctttgctactgctaatggaactgaacccccaggggtattccagttgtaatagcctttccttactgttgtttggttctgtgaatgcctatgttattgatatgtggagggccggaattcttttgcta

SEQ ID NO: 6 - protein, 560 aa, Homo sapiens
MRDVHKDQQLRHTPYSIRCERRMKWHSEDEIRITTWRNRKPPERKMSQNTQDGYTRNWFKVTIPYGIKYDKAWLMNSIQSHCSDRFTPVDFHYVRNRACFFVQDASAASALKDVSYKIYDDENQKICIFVNHSTAPYSVKNKLKPGQMEMLKLTMNKRYNVSQQALDLQNLRFDPDLMGRDIDIILNRRNCMAATLKIIERNFPELLSLNLCNNKLYQLDGLSDITEKAPKVKTLNLSKNKLESAWELGKVKGLKLEELWLEGNPLCSTFSDQSAYVSAIRDCFPKLLRLDGRELSAPVIVDIDSSETMKPCKENFTGSETLKHLVLQFLQQYYSIYDSGDRQGLLGAYHDEACFSLAIPFDPKDSAPSSLCKYFEDSRNMKTLKDPYLKGELLRRTKRDIVDSLSALPKTQHDLSSILVDVWCQTERMLCFSVNGVFKEVEGQSQGSVLAFTRTFIATPGSSSSLCIVNDELFVRDASPQETQSAFSIPVSTLSSSSEPSLSQEQQEMVQAFSAQSGMKLEWSQKCLQDNEWNYTRAGQAFTMLQTEGKIPAEAFKQIS

SEQ ID NO: 7 - protein, 374 aa, Homo sapiens
MSESQDEVPDEVENQFILRLPLEHACTVRNLARSQSVKMKDKLKIDLLPDGRHAVVEVEDVPLAAKLVDLPCVIESLRTLDKKTFYKTADISQMLVCTADGDIHLSPEEPAASTDPNIVRKKERGREEKCVWKHGITPPLKNVRKKRFRKTQKKVPDVKEMEKSSFTEYIESPDVENEVKRLLRSDAEAVSTRWEVIAEDGTKEIESQGSIPGFLISSGMSSHKQGHTSSEYDMLREMFSDSRSNNDDDEDEDDEDEDEDEDEDEDKEEEEEDCSEEYLERQLQAEFIESGQYRANEGTSSIVMEIQKQIEKKEKKLHKIQNKAQRQKDLIMKVENLTLKNHFQSVLEQLELQEKQKNEKLISLQEQLQRFLKK

SEQ ID NO: 8 - protein, 183 aa, Homo sapiens
MATAQPSQVRQKYDTNCDAAINSHITLELYTSYLYLSMAFYFNRDDVALENFFRYFLRLSDDKMEHAQKLMRLQNLRGGHICLHDIRKPECQGWESGLVAMESAFHLEKNVNQSLLDLYQLAVEKGDPQLCHFLESHYLHEQVKTIKELGGYVSNLRKICSPEAGLAEYLFDKLTLGGRVKET

SEQ ID NO: 9 - protein, 2789 aa, Homo sapiens
MPSDAKDSVNGDLLLNWTSLKNILSGLNASFPLHNNTGSSTVTTSKSIKDPRLMRREESMGEQSSTAGLNEVLQFEKSSDNVNSEIKSTPSNSASSSEVVPGDRAVLTNGLDTPCFKTSVNDSQSWAHNMGSEDYDCIPPNKVTMAGQCKDQGNFSFPISVSNVVSEVENQNHSEEKAQRAQQESGNAYTKEYSSHIFQDSQSSDLKTIYQTGCQTSTVFPLKKKVSIDEYLQNTGKMKNFADLEDSSKHEEKQTSWKEIDNDFTNETKISPIDNYIVLHQEYKESESHNSFGKSCDKILITQELEITKSSTSTIKDKDELDHLALEWQITPSFESLSQKHPQHSVEYEGNIHTSLAIAQKLMELKLGKINQNYASIITEAFPKPKDIPQAKEMFIDTVISSYNIETAHDSSNCSITREHICVHRKNENEPVSLENIQRDYKETAYVEDRGQDHNLFCNSQLSNDIWLNVNFKKQTDRENQNEAKENSASCVENNIENIYGDKKQDSHTNENFSNIDEKEDKNYHNIEILSSEEFSTKFNLICREDNAVSAATALLESEEDTISAVKQKDTENTGRSVEHLASTTFPKTASSSVCVASNAAIQIASATMPALSLNNDDHQIYQFKETCSSESPDFGLLVKHRVSDCEIDTDKNKSQESFHQSINENLVLQSIELESEIEIELEDCDDAFIFQQDTHSHENMLCEEFVTSYKALKSRISWEGLLALDNGEMEVLESTTGRENSDQHYSKESNYFYSSTQNNETELTSPILLPDLQIKITNIFRPGFSPTADSLALKDSFCTHVTEATKPEINKEDGEILGFDIYSQPFGENADYPCEDKVDNIRQESGPVSNSEISLSFDLSRNTDVNHTSENQNSESLFTEPSNVTTIDDGSRCFFTKSKTDYNDTKNKKEVESRISKRKLHISSRDQNIPHKDLRRHKIYGRKRRLTSQDSSECFSSLSQGRIKTFSQSEKHIKSVLNILSDEASLCKSKCLSRKLDKAVVHLKKAHRRVHTSLQLITKVGEERKGPLPKSYAVICNNFWESCDLQGYSSVSQRKYYSTKHFSSKRKYDKRRKKRAPKADISKSLTHVSKHKSYKTSGEKKCLSRKSMASSVSKSHPTTSHMGEFCNQEHPESQLPVSSTSQSTSQSVYYNSSVSNPSLSEEHQPFSGKTAYLFSPDHSDEKLIEKENQIDTAFLSSTSKYEKLEKHSANHNVKDATKENSCDANEVINESNSVSLSCIKENINSSTGNDCDATCIGHTKAKTDVLISVLDSNVKHFLNDLYQQGNLILSDCKRNLEVKWTDPIERPKQSIITGNFLMGPLNLTLIASKKYSIPQVSAAAVTDSEGESSKSYLDKQRILTVDSFAASSTVPHCEQSCREKELLKTEQCSSGNCLHTDGNETNVTENYELDVASGTEEDKSYGENIVELSSSDSSLLLRDNVKGSSSETCIVKKDTEDRITWKVKQAEKAKDSVYKRSMTEGSTVNTEYKNQKNQISEESCLNEKIITTNLIDSHLSTKNTTTESVPLKNTVSNPLNKREKKGEIKVSKDSQSDLTLHSEIAYISKPGILGVNHTPILPAHSETCKVPTLLKKPASYVSDFKEKHCSANHTALIANLSQILQRADEASSLQILQEETKVCLNILPLFVEAFERKQECSVEQILISRELLVDQNLWNNCKHTLKPCAVDTLVELQMMMETIQFIENKKRHLEGEPTLRSLLWYDETLYAELLGKPRGFQQQSNFYPGFQGRLKYNAFCELQTYHDQLVELLEETKREKNSYYVFLKYKRQVNECEAIMEHCSDCFDFSLSVPFTCGVNFGDSLEDLEILRKSTLKLINVCGDSPKVHSYPGKQDHLWIIIEMISSKVNFIKNNEAVRVKISLYGLEHIFFDAAKNLVWKERTQSFSKKYSQKKDEERLLRVNKCAFSKLQKIYDTLSKDLNNEPISPIGLEEDTIIASRKSDHPINEATISIENSKFNSNLLAHPDICCISEILDQAEFADLKKLQDLTLRCTDHLEILKKYFQMLQDNNMDNIFITEENVLDVVINHSHEAIILKPEAIEMYIEIVMVSETIHFLKNSIAKKLDKQRFRGMLWFDLSLLPELVQCQEKMASFSFLKDNSTDVCLWKVIETAVSELKKDLDIICKYNEAVNCSYAIHLLSRELQELSEIKKLLKKSKYFISTYIDFVPYIASINYGSTVTELEYNYNQFSTLLKNVMSAPRKDLGKMAHIRKVMKTIEHMKMICTKNAELTISFFLCQMLYNRRKILQLKRKEKMNIHIVKPGENNNKFSISTMLPPVSECINKNISNSSKKRPSTVDKCEDSQEQQQDTTVSSCKKLKVDMKDVTKINREKATFKHPRTTGSHPKSENKIVPSSCDSLKRNHLTPKKVEMQRSLPGSLLPLENPKDTCASKSESKIDLTVSSDHFSGQQENLNSMKKRNVNFSAAETKSDKKDCAAFAICDQKSVHGTFSPDHGTLLQKFLKNSPDPTQKSCLSDINPETDVSLVPDASVLSKPIFCFVKDVHPDLEMNDTVFELQDNDIVNSSIKNSSCMTSPEPICIQNKIPTLQINKLQPTETESEDKYMKDTLNPNTVHTFGASGHITLNVNQGAEYSLSEQQNDKNSKVLMQNAATYWNELPQSACNPTYNSSEHLFGTSYPYSAWCVYQYSNSNGNAITQTYQGITSYEVQPSPSGLLTTVASTAQGTHSNLLYSQYFTYFAGEPQANGFVPVNGYFQSQIPASNFRQPIFSQYASHQPLPQATYPYLPNRFVPPEVPWVYAPWHQESFHPGH

SEQ ID NO: 10 - protein, 777 aa, Homo sapiens
MEQYCSIKIVDILEEEVVTFAVEVELPNSGKLLDHVLIEMGYGLKPSGQDSKKENADQSDPEDVGKMTTENNIVVDKSDLIPKVLTLNVGDEFCGVVAHIQTPEDFFCQQLQSGRKLAELQASLSKYCDQLPPRSDFYPAIGDICCAQFSEDDQWYRASVLAYASEESVLVGYVDYGNFEILSLMRLCPIIPKLLELPMQAIKCVLAGVKPSLGIWTPEAICLMKKLVQNKIITVKVVDKLENSSLVELIDKSETPHVSVSKVLLDAGFAVGEQSMVTDKPSDVKETSVPLGVEGKVNPLEWTWVELGVDQTVDVVVCVIYSPGEFYCHVLKEDALKKLNDLNKSLAEHCQQKLPNGFKAEIGQPCCAFFAGDGSWYRALVKEILPNGHVKVHFVDYGNIEEVTADELRMISSTFLNLPFQGIRCQLADIQSRNKHWSEEAITRFQMCVAGIKLQARVVEVTENGIGVELTDLSTCYPRIISDVLIDEHLVLKSASPHKDLPNDRLVNKHELQVHVQGLQATSSAEQWKTIELPVDKTIQANVLEIISPNLFYALPKGMPENQEKLCMLTAELLEYCNAPKSRPPYRPRIGDACCAKYTSDDFWYRAVVLGTSDTDVEVLYADYGNIETLPLCRVQPITSSHLALPFQIIRCSLEGLMELNGSSSQLIIMLLKNFMLNQNVMLSVKGITKNVHTVSVEKCSENGTVDVADKLVTFGLAKNITPQRQSALNTEKMYRTNCCCTELQKQVEKHEHILLFLLNNSTNQNKFIEMKKLVKS

SEQ ID NO: 11 - DNA, 18 bp, Homo sapiens
ctattcccttcgacccca

SEQ ID NO: 12 - DNA, 21 bp, Homo sapiens
ctctttgggtggttatgtcac

SEQ ID NO: 13 - DNA, 22 bp, Homo sapiens
cactaaggacccctacctgaag

SEQ ID NO: 14 - DNA, 21 bp, Homo sapiens
gaagtaaagagactgctgcgt

SEQ ID NO: 15 - DNA, 21 bp, Homo sapiens
cgagtggggtctttaagtcta

SEQ ID NO: 16 - DNA, 23 bp, Homo sapiens
agaaagtcaaggctccatcccag

SEQ ID NO: 17 - DNA, 19 bp, Homo sapiens
tggccctggagaacttctt

SEQ ID NO: 18 - DNA, 20 bp, Homo sapiens
atggtcttgacttgctcgtg

SEQ ID NO: 19 - DNA, 20 bp, Homo sapiens
tggccacatctgccttcacg

SEQ ID NO: 20 - DNA, 21 bp, Homo sapiens
gtctgcatgtaacccaacata

SEQ ID NO: 21 - DNA, 20 bp, Homo sapiens
gtgtcctggatgaaaggatt

SEQ ID NO: 22 - DNA, 23 bp, Homo sapiens
tcagtacagcaacagcaatggca

SEQ ID NO: 23 - DNA, 21 bp, Homo sapiens
ggtagttggtatcgtgcttta

SEQ ID NO: 24 - DNA, 23 bp, Homo sapiens
gaacatcactaattattctggga

SEQ ID NO: 25 - DNA, 21 bp, Homo sapiens
cctttcagggaatacggtgcc

(19) United States
(12) Patent Application Publication
(10) Pub. No.: US 2005/0181370 A1
(43) Pub. Date: Aug. 18, 2005

(54) Cancer-associated nucleic acids and polypeptides

(76) Inventors: Charles De Smet, Brussels (BE); Thierry Boon-Falleur, Brussels (BE); Axelle Loriot, Brussels (BE)

Correspondence Address: WOLF GREENFIELD & SACKS, PC;FEDERAL RESERVE PLAZA, 600 ATLANTIC AVENUE, BOSTON, MA, 02210-2211, US

(21) Appl. No.: 10/512,633

(22) PCT Filed: Apr. 17, 2003
(86) PCT No.: PCT/US03/11933

Related U.S. Application Data
(60) Provisional application No. 60/375,879, filed on Apr. 26, 2002.

(51) Int. Cl. C12Q 1/68

(52) U.S. Cl. 435/6

(57) ABSTRACT

The invention provides methods of diagnosing cancer, based on the identification of expression of certain-associated nucleic acids/or polypeptides. The identified nucleic acids and/or polypeptides can be utilized as markers for diagnosing cancer, and for following the course of treatment of cancer.

### RELATED APPLICATIONS

This application claims priority under 35 U.S.C. §119 from U.S. provisional application Ser. No. 60/375,879, filed Apr. 26, 2002.

### FIELD OF THE INVENTION

The invention relates to use of one or more cancer-associated nucleic acid molecules and the polypeptides they encode as markers for cancer. The invention also relates to the use of cancer-associated nucleic acid molecules and the polypeptides they encode in methods of diagnosing and treating cancer. In addition, the invention relates to the use of such nucleic acid molecules and the polypeptides they encode for monitoring the cancer's response to treatment.

### BACKGROUND OF THE INVENTION

An element in the successful treatment of cancer is the selection and implementation of an appropriate combination of therapeutic approaches. For example, depending on a cancer patient's prognosis, therapy may include surgical intervention in combination with adjuvant therapy or it may only include surgical intervention. In addition, for some patients pretreatment with chemotherapy or radiotherapy is utilized prior to surgical intervention, but in other patients adjuvant therapies are used following surgical intervention.

Determination of appropriate treatment for an individual cancer patient is complex with a wide variety of treatments and possible treatment combinations. For example, chemotherapy is a common method of cancer treatment, with more than 50 different chemotherapeutic agents available. These therapeutic agents can be used in a wide range of dosages both singly and in combinational therapies with other chemotherapeutic agents, surgery, and/or radiotherapy. The available methods for designing strategies for treating cancer patients are complex and inexact. Therefore it is important to monitor the impact of the treatment on the cancer. By monitoring the effectiveness of a treatment strategy, the treatment can be modified as necessary to improve the chances for long-term patient survival.

Because of the importance of timely and accurate cancer diagnosis, as well as the importance of selecting appropriate treatment regimens for cancer patients and for following their progress, the development of methods to detect cancer or the potential for cancer and to monitor treatment are of key interest to those in the medical community and their patients. Although available diagnostic procedures for cancer may be partially successful, the methods for detecting cancer and monitoring its treatment remain unsatisfactory. There is a critical need for diagnostic tests that can detect cancer at its early stages, when appropriate treatment may substantially increase the likelihood of positive outcome for the patient. It is also important that cancer treatment be monitored to allow the treatment to be adapted as necessary to best serve the patient's clinical needs. Such diagnostic and monitoring methods would enable medical care professionals to identify cancer, select optimal treatment regimens for individual patients, and to assess the cancer before, during, and after treatment.

### SUMMARY OF THE INVENTION

Surprisingly, it has now been found that certain nucleic acids with expression that is substantially restricted to testis are also expressed in cancerous tissues. The identification of this selective expression allows use of these cancer-associated nucleic acids, or the polypeptides they encode, in cancer diagnostic assays and kits. Such methods, assays and kits are useful to detect cancer in human subjects, and for staging cancer in subjects. The methods, assays, and kits described herein may also be used in treatments for cancer and to evaluate treatments for cancer.

According to one aspect of the invention, methods for diagnosing cancer in a subject are provided. The methods include obtaining a non-testis biological sample from a subject, determining the expression in the sample of one or more cancer-associated nucleic acid molecules comprising a nucleotide sequence selected from the group consisting of SEQ ID NO: 1-5, wherein if the nucleotide sequence is SEQ ID NO: 1, the biological sample is not a tracheal sample, wherein if the nucleotide sequence is SEQ ID NO: 4, the biological sample is not a brain, ovarian, or uterine sample, wherein if the nucleotide sequence is SEQ ID NO: 5, the biological sample is not a brain, ovarian, thymus, spleen, colon, or tracheal sample, and wherein expression of the nucleic acid molecule in the sample is diagnostic for cancer in the subject. In some embodiments, expression is determined for at least 2, 3, 4, or 5 nucleic acid molecules comprising a nucleotide sequence selected from the group consisting of SEQ ID NOs:1-5. In certain embodiments, the sample is selected from the group consisting of: tissue and cells. In certain embodiments, the expression of cancer-associated nucleic acid molecules is determined by a method selected from the group consisting of nucleic acid hybridization and nucleic acid amplification. In some embodiments, the hybridization is performed using a nucleic acid microarray. In some embodiments, the nucleic acid amplification is RT-real-time PCR or RT-PCR.

According to another aspect of the invention, methods for diagnosing cancer in a subject are provided. The methods include obtaining a non-testis biological sample from a subject, determining the level of expression of a cancer-associated nucleic acid molecule comprising one or more nucleotide sequences selected from the group consisting of SEQ ID NOs: 1-5, comparing the level of expression of the nucleic acid molecule in the subject sample to a level of expression of the nucleic acid in a testis control tissue, wherein a determination that the level of expression of the nucleic acid in the sample from the subject is greater than about 1.8% of the level of expression of the nucleic acid in the control tissue, is diagnostic for cancer in the subject. In some embodiments, the level of expression of the nucleic acid in the sample from the subject is at least about 1.9%, 2.0%, 2.5%, 3.0%, 4%, 5%, 10%, 20%, 30%, 40%, 50%, 60%, 70%, 80%, or 90% of the level of expression in the control tissue. In certain embodiments, expression is determined for at least 2, 3, 4, or 5 nucleic acid molecules comprising a nucleotide sequence selected from the group consisting of SEQ ID NOs:1-5. In some embodiments, the sample is selected from the group consisting of: tissue and cells. In some embodiments, the expression of cancer-associated nucleic acid molecules is determined by a method selected from the group consisting of nucleic acid hybridization and nucleic acid amplification. In some embodiments, the hybridization is performed using a nucleic acid microarray. In certain embodiments, the nucleic acid amplification is RT-real-time PCR or RT-PCR.

According to another aspect of the invention, methods for determining onset, progression, or regression, of cancer in a subject are provided. The methods include obtaining from a subject two non-testis biological samples, wherein the samples comprise the same tissue type and are obtained at different times, determining a level of expression of one or more cancer-associated nucleic acid molecules or expression products thereof in the two samples, wherein the nucleic acid molecules comprise nucleotide sequences selected from the group consisting of: SEQ ID NOs: 1-5, wherein if the nucleotide sequence is SEQ ID NO: 1, the biological sample is not a tracheal sample, wherein if the nucleotide sequence is SEQ ID NO: 4, the biological sample is not a brain, ovarian, or uterine sample, wherein if the nucleotide sequence is SEQ ID NO: 5, the biological sample is not a brain, ovarian, thymus, spleen, colon, or tracheal sample. The methods also include comparing the levels of expression in the two samples wherein a higher level of expression of the one or more cancer-associated nucleic acid molecules in the first non-testis sample than in the second non-testis sample indicates regression of cancer, wherein a lower level of expression of the one or more cancer-associated nucleic acid molecules in the first non-testis sample than the second non-testis sample indicates progression of cancer, and wherein a level of expression of the one or more cancer-associated nucleic acid molecules in the first non-testis sample that is less than the level of expression of the cancer-associated nucleic acid molecules in the second testis sample indicates onset of cancer. In some embodiments, expression is determined for at least 2, 3, 4, or 5 nucleic acid molecules comprising a nucleotide sequence selected from the group consisting of SEQ ID NOs:1-5. In certain embodiments, the expression products are polypeptides comprising amino acid sequences selected from the group consisting of SEQ ID NOs: 6-10. In some embodiments, the expression of the expression products is determined by hybridization. In certain embodiments, the hybridization is performed with a microarray. In some embodiments, the expression of cancer-associated nucleic acid molecules is determined by a method selected from the group consisting of nucleic acid hybridization and nucleic acid amplification. In certain embodiments, the hybridization is performed using a nucleic acid microarray. In some embodiments, the nucleic acid amplification is RT-real-time PCR or RT-PCR.

According to another aspect of the invention, methods for determining onset, progression, or regression, of cancer in a subject are provided. The methods include obtaining from a subject a first and a second non-testis biological sample, wherein the samples comprise the same tissue type and are obtained at different times, determining a level of expression of one or more cancer-associated nucleic acid molecules or expression products thereof in the first and second non-testis biological samples, wherein the nucleic acid molecules comprise nucleotide sequences selected from the group consisting of: SEQ ID NOs: 1-5, comparing the level of expression of one or more cancer-associated nucleic acid molecules of the first and the second non-testis biological samples to the level of expression of the one or more cancer-associated nucleic acid molecules in a testis control sample, wherein a determination that the level of the one or more cancer-associated nucleic acid molecules in either the first or second non-testis biological samples is more than 1.8% of the level of expression of the one or more cancer-associated nucleic acid molecules in the testis control sample, is an diagnostic for cancer, wherein a higher level of expression of the one or more cancer-associated nucleic acid molecules in the first non-testis sample than in the second non-testis sample indicates regression of cancer, wherein a lower level of expression of the one or more cancer-associated nucleic acid molecules in the first non-testis sample than the second non-testis sample indicates progression of cancer, and wherein a level of expression of the one or more cancer-associated nucleic acid molecules in the first non-testis sample that is less than the level of expression of the one or more cancer-associated nucleic acid molecules in the control testis sample and the level of expression of the cancer-associated nucleic acid molecules in the second testis sample indicates onset of cancer. In some embodiments, the level of expression of the one or more nucleic acid molecules in the sample from the subject is at least about 1.9%, 2.0%, 2.5%, 3.0%, 4%, 5%, 10%, 20%, 30%, 40%, 50%, 60%, 70%, 80%, or 90% of the level of expression in the control tissue. In certain embodiments, expression is determined for at least 2, 3, 4, or 5 nucleic acid molecules comprising a nucleotide sequence selected from the group consisting of SEQ ID NOs:1-5. In some embodiments, the sample is selected from the group consisting of: tissue and cells. In some embodiments, the expression of cancer-associated nucleic acid molecules is determined by a method selected from the group consisting of nucleic acid hybridization and nucleic acid amplification. In some embodiments, the hybridization is performed using a nucleic acid microarray. In certain embodiments, the nucleic acid amplification is RT-real-time PCR or RT-PCR.

According to another aspect of the invention, methods for diagnosing cancer in a subject are provided. The methods include obtaining a non-testis biological sample from a subject, contacting the sample with antibodies or antigen-binding fragments thereof, that bind specifically to one or more different cancer-associated polypeptides encoded by nucleic acid molecules comprising a nucleotide sequence selected from the group consisting of SEQ ID NOs: 1-5, wherein if the nucleotide sequence is SEQ ID NO: 1, the biological sample is not a tracheal sample, wherein if the nucleotide sequence is SEQ ID NO: 4, the biological sample is not a brain, ovarian, or uterine sample, wherein if the nucleotide sequence is SEQ ID NO: 5, the biological sample is not a brain, ovarian, thymus, spleen, colon, or tracheal sample, and wherein specific binding of one or more antibodies or antigen-binding fragments thereof in the sample is diagnostic for cancer in the subject. In some embodiments, specific binding is determined for at least 2, 3, 4, or 5 polypeptides encoded by nucleic acid molecules comprising a nucleotide sequence selected from the group consisting of SEQ ID NOs:1-5. In certain embodiments, the sample is selected from the group consisting of: tissue and cells. In some embodiments, the specific binding of cancer-associated polypeptides is determined by immunohistochemistry.

According to yet another aspect of the invention, methods for diagnosing cancer in a subject are provided. The methods include obtaining a non-testis biological sample from a subject, contacting the sample with antibodies or antigen-binding fragments thereof, that bind specifically to one or more different cancer-associated polypeptides encoded by nucleic acid molecules comprising a nucleotide sequence selected from the group consisting of SEQ ID NOs:1-5, determining the level of specific binding between the antibodies or antigen-binding fragments thereof and cancer-associated polypeptides in the sample, comparing the level of specific binding between the antibodies or antigen-binding fragments thereof and cancer-associated polypeptides in a testis control tissue, wherein a determination that the level of specific binding in the sample from the subject is more than 1.8% of the level of specific binding in the control tissue, is diagnostic for cancer in the subject. In some embodiments, expression is determined for at least 2, 3, 4, or 5 polypeptides encoded by nucleic acid molecules comprising a nucleotide sequence selected from the group consisting of SEQ ID NOs:1-5. In certain embodiments, the sample is selected from the group consisting of: tissue and cells. In some embodiments, the expression of cancer-associated polypeptides is determined by a method selected from the group consisting of immunohistochemistry and hybridization. In some embodiments, the hybridization is performed using a microarray.

According to yet another aspect of the invention, methods for determining onset, progression, or regression, of cancer in a subject are provided. The methods include obtaining from a subject two non-testis biological samples, wherein the samples comprise the same tissue type and are obtained at different times, contacting the two samples with antibodies or antigen-binding fragments thereof, that bind specifically to one or more cancer-associated polypeptides encoded by nucleic acid molecules comprising a nucleotide sequence selected from the group consisting of SEQ ID NO:s: 1-5, determining a level of specific binding between the one or more cancer-associated polypeptides in the at least two samples, wherein if the nucleotide sequence is SEQ ID NO: 1, the biological sample is not a tracheal sample, wherein if the nucleotide sequence is SEQ ID NO: 4, the biological sample is not a brain, ovarian, or uterine sample, wherein if the nucleotide sequence is SEQ ID NO: 5, the biological sample is not a brain, ovarian, thymus, spleen, colon, or tracheal sample, and comparing the levels of specific binding of the one or more cancer-associated polypeptides in the at least two samples to determine the onset, progression, or regression of the cancer, wherein a higher level of specific binding of the one or more cancer-associated polypeptides in the first sample than in the second sample indicates regression of cancer, wherein a lower level of specific binding of the one or more cancer-associated polypeptides in the first sample than the second non-testis sample indicates progression of cancer, and wherein a level of specific binding of the one or more cancer-associated polypeptides in the first non-testis sample that is less than the level of expression of the cancer-associated nucleic acid in the second testis sample indicates onset of cancer. In some embodiments, the sample is selected from the group consisting of: tissue and cells. In certain embodiments, binding is determined between the cancer-associated polypeptides and antibodies or antigen-binding fragments thereof, that bind specifically to least 2, 3, 4, or 5 different cancer-associated polypeptides encoded by nucleic acid molecules comprising a nucleotide sequence selected from the group consisting of SEQ ID NOs:1-5. In some embodiments, the antibodies are monoclonal or polyclonal antibodies. In some embodiments, the antibodies are chimeric, human, or humanized antibodies. In certain embodiments, the antibodies are single chain antibodies. In some embodiments, the antigen-binding fragments are F(ab′)₂, Fab, Fd, or Fv fragments.

According to yet another aspect of the invention, methods for determining onset, progression, or regression, of cancer in a subject are provided. The methods include obtaining from a subject two non-testis biological samples, wherein the samples comprise the same tissue type and are obtained at different times, contacting the two samples with antibodies or antigen-binding fragments thereof, that bind specifically to one or more cancer-associated polypeptides encoded by nucleic acid molecules comprising a nucleotide sequence selected from the group consisting of SEQ ID NO:s: 1-5, determining a level of specific binding between the one or more cancer-associated polypeptides in the at least two samples, comparing the levels of specific binding of the one or more cancer-associated polypeptides in the two samples to the level of specific binding of the one or more cancer-associated polypeptides in a testis control sample, wherein a determination that the level of specific binding of the one or more cancer-associated polypeptides in either the first or second non-testis sample is more than 1.8% of the level of specific binding of the one or more cancer associated polypeptides in the testis control sample, is diagnostic for cancer, wherein a higher level of specific binding of the one or more cancer-associated polypeptides in the first non-testis sample than the second non-testis sample indicates regression of cancer, wherein a lower level of specific binding of the one or more cancer-associated polypeptides in the first non-testis sample than the second non-testis sample indicates progression of cancer, and wherein a level of specific binding of the one or more cancer-associated polypeptides in the first non-testis sample that is less than the level of specific binding of the one or more cancer-associated polypeptides in a control testis sample and is less than the level of binding of the one or more cancer-associated polypeptides in the second testis sample indicates onset of cancer. In some embodiments, the sample is selected from the group consisting of: tissue and cells. In certain embodiments, binding is determined between the cancer-associated polypeptides and antibodies or antigen-binding fragments thereof, that bind specifically to least 2, 3, 4, or 5 different cancer-associated polypeptides encoded by nucleic acid molecules comprising a nucleotide sequence selected from the group consisting of SEQ ID NOs:1-5. In some embodiments, the antibodies are monoclonal or polyclonal antibodies. In some embodiments, the antibodies are chimeric, human, or humanized antibodies. In some embodiments, the antibodies are single chain antibodies. In some embodiments, the antigen-binding fragments are F(ab′)₂, Fab, Fd, or Fv fragments.

According to another aspect of the invention, methods for treating a subject with a disorder characterized by increased expression of a cancer-associated nucleic acid molecule are provided. The methods include administering to the subject an effective amount of an antisense molecule to a cancer-associated nucleic acid molecule comprising a nucleotide sequence selected from the group consisting of SEQ ID NOs:1-5 to treat the cancer.

According to another aspect of the invention, methods for treating a subject with cancer characterized by increased expression or activity of a cancer-associated polypeptide are provided. The methods include administering to the subject an effective amount of an antibody or an antigen-binding fragment thereof to a cancer-associated polypeptide comprising an amino acid sequence selected from the group consisting of SEQ ID NOs:6-10 to treat the cancer. In some embodiments the antibodies or antigen-binding fragments are labeled with one or more cytotoxic agents. In certain embodiments, the antibodies are monoclonal or polyclonal antibodies. In some embodiments, the antibodies are chimeric, human, or humanized antibodies. In certain embodiments, the antibodies are single chain antibodies. In some embodiments, the antigen-binding fragments are F(ab′)₂, Fab, Fd, or Fv fragments.

According to yet another aspect of the invention, methods for selecting a course of treatment of a subject having or suspected of having cancer are provided. The methods include obtaining from the subject a biological sample, contacting the sample with antibodies or antigen-binding fragments thereof that bind specifically to one or more different cancer-associated polypeptides encoded by nucleic acid molecules comprising a nucleotide sequence selected from the group consisting of SEQ ID NOs:1-5, wherein if the nucleotide sequence is SEQ ID NO: 1, the biological sample is not a tracheal sample, wherein if the nucleotide sequence is SEQ ID NO: 4, the biological sample is not a brain, ovarian, or uterine sample, wherein if the nucleotide sequence is SEQ ID NO: 5, the biological sample is not a brain, ovarian, thymus, spleen, colon, or tracheal sample, determining specific binding between cancer-associated polypeptides in the sample that are differentially expressed in different types of cancer, and the antibodies or antigen-binding fragments thereof, and selecting a course of treatment appropriate to the cancer of the subject. In some embodiments, the treatment is administering antisense nucleic acid molecules that specifically bind to cancer-associated nucleic acid molecules. In certain embodiments, the treatment is administering antibodies that specifically bind to the cancer-associated polypeptides. In some embodiments, the antibodies are labeled with one or more cytotoxic agents. In some embodiments, the sample is selected from the group consisting of: tissues and cells. In some embodiments, binding is determined between the cancer-associated polypeptides and antibodies or antigen-binding fragments thereof, that bind specifically to least 2, 3, 4, or 5 different cancer-associated polypeptides encoded by nucleic acid molecules comprising a nucleotide sequence selected from the group consisting of SEQ ID NOs:1-5. In certain embodiments, the antibodies are monoclonal or polyclonal antibodies. In some embodiments, the antibodies are chimeric, human, or humanized antibodies. In some embodiments, the antibodies are single chain antibodies. In certain embodiments, the antigen-binding fragments are F(ab′)₂, Fab, Fd, or Fv fragments.

According to yet another aspect of the invention, kits for the diagnosis of cancer in a subject are provided. The kits include one or more antibodies or antigen-binding fragments thereof that bind specifically to cancer-associated polypeptides encoded by nucleic acid molecules comprising a nucleotide sequence selected from the group consisting of SEQ ID NOs:1-5, one or more control agents, and instructions for the use of the agents in the diagnosis of cancer. In some embodiments, the one or more agents are antibodies or antigen-binding fragments thereof. In certain embodiments, the one or more agents are bound to a substrate. In some embodiments, the kit includes antibodies or antigen-binding fragments thereof, that bind specifically to least 2, 3, 4, or 5 cancer-associated polypeptides encoded by nucleic acid molecules comprising a nucleotide sequence selected from the group consisting of SEQ ID NOs:1-5.

According to another aspect of the invention, methods for diagnosing cancer in a subject are provided. The methods include contacting a non-testis tissue in a subject with an antibody that selectively binds to a cancer-associated polypeptide comprising an amino acid sequence selected from the group consisting of SEQ ID NOs:6-10, determining the binding in the tissue of the antibody, wherein binding of the antibody in the tissue is diagnostic for cancer in the subject. In some embodiments, the antibody is labeled with a fluorescent or radioactive label. In certain embodiments, the antibody binding is detected with in vivo imaging methods.

In some embodiments of the foregoing methods, the sample is selected from the group consisting of: tissue and cells. In some embodiments, the tissue is selected from the group consisting of: colorectal, lung, breast, bladder, epidermis, thyroid, cutaneous, neuronal, prostate, esophageal, renal, spleen, blood, and bone marrow. In some embodiments, the cancer is selected from the group consisting of: cutaneous melanomas, sarcomas, lung carcinomas, bladder carcinomas, epidermoid carcinoma, renal tumors, breast carcinomas, prostate tumors, esophageal carcinomas, leukemias, uterine tumors, brain tumors, thyroid tumors, neuroblastomas, colorectal carcinomas, mesotheliomas, uveal melanomas, and myelomas.

### DETAILED DESCRIPTION OF THE INVENTION

The invention described herein relates in part to the novel identification of selective expression of nucleic acids and the polypeptides they encode in tissues with cancer, including, but not limited to: colorectal, lung, breast, bladder, epidermis, thyroid, brain, prostate, esophageal, renal, spleen, blood, and bone marrow. The cancer-associated nucleic acids and the polypeptides they encode may be used as markers for cancers, including, but not limited to: cutaneous melanomas, sarcomas, lung carcinomas, bladder carcinomas, epidermoid carcinoma, renal tumors, breast carcinomas, prostate tumors, esophageal carcinomas, leukemias, uterine tumors, brain tumors, thyroid tumors, neuroblastomas, colorectal carcinomas, mesotheliomas, uveal melanomas, and myelomas. In addition, the cancer-associated nucleic acids and the polypeptides they encode may also be used in methods to treat cancer and in methods to assess the response of cancer to treatment regimens.

As used herein, “cancer-associated nucleic acid molecules” means nucleic acid molecules expressed in tissues that are cancerous. The invention also relates in part to “cancer-associated polypeptides,” which as used herein means polypeptides that are encoded by nucleic acid molecules expressed in tissues that are cancerous. The invention also relates, in part, to the use of the nucleic acid molecules that encode the cancer-associated polypeptides and also relates in part to the use of the cancer-associated polypeptides. In all embodiments, human cancer-associated nucleic acids and polypeptides are preferred.

The cancer-associated nucleic acid molecules disclosed herein (SEQ ID NOs:1-5) encode the cancer-associated polypeptides (SEQ ID NOs:6-10) shown in Table 1.

TABLE 1
Cancer-associated Nucleic acid and Amino Acids

| | Nucleic Acid | |
|---|---|---|
| Gene Name, Accession No. | SEQ ID NO | Amino Acid SEQ ID NO |
| NXF2, AF285596 | 1 | 6 |
| TAF2Q, AF285595 | 2 | 7 |
| FTHL17, AF285592 | 3 | 8 |
| TEX15, AF285605 | 4 | 9 |
| TDRD1, AF285606 | 5 | 10 |

As used herein, a “subject” is preferably a human, non-human primate, cow, horse, pig, sheep, goat, dog, cat, or rodent. In all embodiments, human subjects are preferred. In some embodiments, the subject is suspected of having or has been diagnosed with cancer. Cancers in which the cancer-associated nucleic acid or polypeptide are expressed include: cutaneous melanomas, sarcomas, lung carcinomas, bladder carcinomas, epidermoid carcinoma, renal tumors, breast carcinomas, prostate tumors, esophageal carcinomas, leukemias, uterine tumors, brain tumors, thyroid tumors, neuroblastomas, colorectal carcinomas, mesotheliomas, uveal melanomas, and/or myelomas.

As used herein, “different types” of cancer may include different histological types, and/or different tumor types. For example, different types of breast cancer may include, but are not limited to: invasive ductal carcinoma, invasive pleomorphic lobular carcinoma. As used herein, “different types” of cancer may also mean different cell types and different stages of cancer (e.g., primary tumor or metastatic growth).

Methods for identifying subjects suspected of having cancer may include but are not limited to: manual examination, biopsy, subject's family medical history, subject's medical history, or a number of imaging technologies such as mammography, magnetic resonance imaging, magnetic resonance spectroscopy, or positron emission tomography. The foregoing diagnostic methods for cancer and the clinical delineation of cancer diagnoses are well known to those of skill in the medical arts.

As used herein, a “biological sample” includes, but is not limited to: tissue, cells, or body fluid (e.g. blood or lymph node fluid). The fluid sample may include cells and fluid. The tissue and cells may be obtained from a subject or may be grown in culture (e.g. from a cell line). As used herein, a “biological sample” is tissue or cells obtained (e.g., from a tissue biopsy, aspiration, or fluid collection) using methods well known to those of ordinary skill in the related medical arts. The phrase “suspected of being cancerous” as used herein means a tissue or tissue sample believed by one of ordinary skill in the art to contain cancerous cells. Examples of methods for obtaining the sample from the biopsy include aspiration, gross apportioning of a mass, microdissection, laser-based microdissection, or other art-known cell-separation methods.

As used herein, a “non-testis sample” or “non-testis tissue sample” is a cell or tissue sample from a source that is not testis tissue or does not contain testis tissue. As used herein, a “testis sample”, a “testis biological sample”, or a “testis tissue sample” is a cell or tissue sample from testis tissue or containing testis tissue. In some embodiments of the invention, the testis tissue sample is a control tissue sample, and the level of expression of one of the cancer-associated nucleic acid or polypeptide molecules of the invention on in such testis tissue is a control level of expression.

There may be low-level expression of some cancer-associated nucleic acid molecules or cancer-associated polypeptide molecules of the invention in some normal, non-cancerous tissues (see e.g., Examples 1, 4, and 5). In these tissues a determination of the level of expression of the cancer-associated nucleic acids and/or polypeptides is diagnostic of cancer if the level of expression is above a baseline level determined for that tissue type. The baseline level of expression can be determined using standard methods known to those of skill in the art. Such methods include, for example, assaying a number of histologically normal testis tissue samples from subjects that are clinically normal (i.e. do not have clinical signs of cancer in testis tissue) and determining the mean level of expression for the samples. This baseline level can then be compared to the low level expression of the cancer associated molecules in normal, non-testis tissues, and the comparison represented can be represented as a percentage of the baseline level of expression of the cancer-associated nucleic acid and/or polypeptide in testis control tissue.

Table 2 describes the baseline level of expression of certain cancer-associated nucleic acids and/or polypeptides in some tissues. If the level of expression of a cancer-associated nucleic acid or polypeptide in a tissue is above the baseline level for that tissue and cancer-associated nucleic acid and/or polypeptide, then the tissue is diagnosed as cancerous. For example, a determination that the level of expression of the cancer-associated nucleic acid molecule TEX15 (SEQ ID NO: 4) in an ovary tissue sample is greater than about 1.8% of the level of expression of the cancer-associated nucleic acid molecule in a control testis tissue sample indicates cancer in the ovary tissue sample.

TABLE 2
Baseline levels of expression

| NXF2 SEQ | TEX15, SEQ | TDRD1, SEQ |
|---|---|---|
| ID NOs: 1, 6 | ID NOs: 4, 9 | ID NOs: 5, 10 |
| Trachea 1% | Brain 1.5% | Brain 0.3% |
| | Ovary 1.8% | Colon 0.9% |
| | Uterus 0.5% | Ovary 0.3% |
| | | Spleen 0.2% |
| | | Thymus 0.3% |
| | | Trachea 1.5% |

Tissues that may have low-level expression of cancer-associated nucleic acids of the invention include, but are not limited to: low-level expression of SEQ ID NO: 1 and the polypeptide it encodes in tracheal tissue, low-level expression of SEQ ID NO: 4 and the polypeptide it encodes in brain, ovarian, or uterine tissue, and low-level expression of SEQ ID NO: 5 and the polypeptide it encodes in brain, ovarian, thymus, spleen, colon, or tracheal tissue. Thus, in some tissues there is a baseline level of expression of a cancer-associated molecule of the invention, and it is that baseline level that determines the level above which expression indicates cancer in the tissue. Therefore, as described herein, in some tissues, the level of expression of the nucleic acid molecules of the invention or the polypeptides they encode indicate cancer in the tissue when the level of expression of the nucleic acid molecule is greater than about 0.1% of that in a control testis tissue sample. A level of expression of greater than about 0.1%, 0.15%, 0.2%, 0.3%, 0.4%, 0.5%, 0.6%, 0.7%, 0.8%, 0.9%, 1.0%, 1.1% 1.2% 1.3% 1.4% 1.5%, 1.6% 1.7% 1.8% 1.9%, 2.0%, 2.1%, 2.2%, 2.3%, 2.4%, 2.5%, 2.6%, 2.7%, 2.8%, 2.9%, 3.0%, 4%, 5%, 10%, or more of the level of expression in the control tissue indicates cancer in the tissue.

The invention involves in some aspects diagnosing or monitoring cancer by determining the level of expression of one or more cancer-associated nucleic acid molecules and/or determining the level of expression of one or more cancer-associated polypeptides they encode. In some important embodiments, this determination is performed by assaying a tissue sample from a subject for the level of expression of one or more cancer-associated nucleic acid molecules or for the level of expression of one or more cancer-associated polypeptides encoded by the nucleic acid molecules of the invention.

The expression of the molecules of the invention may be determined using routine methods known to those of ordinary skill in the art. These methods include, but are not limited to: direct RNA amplification, reverse transcription of RNA to cDNA, real-time RT-PCR, amplification of cDNA, hybridization, and immunologically based assay methods, which include, but are not limited to immunohistochemistry, antibody sandwich capture assay, ELISA, and enzyme-linked immunospot assay (EliSpot assay). For example, the determination of the presence of level of nucleic acid molecules of the invention in a subject or tissue can be carried out via any standard nucleic acid determination assay, including the polymerase chain reaction, or assaying with labeled hybridization probes. Such hybridization methods include, but are not limited to microarray techniques.

These methods of determining the presence and/or level of the molecules of the invention in cells and tissues may include use of labels to monitor the presence of the molecules of the invention. Such labels may include, but are not limited to radiolabels or chemiluminescent labels, which may be utilized to determine whether a molecule of the invention is expressed in a cell or tissue, and to determine the level of expression in the cell or tissue. For example, a fluorescently labeled or radiolabeled antibody that selectively binds to a polypeptide of the invention may be contacted with a tissue or cell to visualize the polypeptide in vitro or in vivo. These and other in vitro and in vivo imaging methods for determining the presence of the nucleic acid and polypeptide molecules of the invention are well known to those of ordinary skill in the art.

The invention thus involves in one aspect, the identification of expression of cancer-associated polypeptides in cancerous cells in tissues, genes encoding those polypeptides, functional modifications and variants of the foregoing, useful fragments of the foregoing, as well as diagnostics relating thereto, and diagnostic and therapeutic uses thereof. The cancer-associated polypeptide genes correspond to SEQ ID NOs: 1-5. Encoded polypeptides (e.g., proteins), peptides and antisera thereto are also preferred for diagnosis and correspond to SEQ ID NOs: 6-10.

The amino acid sequences identified herein as cancer-associated polypeptides, and the nucleotide sequences encoding them, are sequences deposited in databases such as GenBank. The discovery that these nucleic acids and the polypeptides are expressed in cancer is unexpected. The identification of these selectively expressed cancer-associated molecules of the invention provides a basis for methods of diagnosing and treating cancer, therapeutic pharmaceutical agents and compounds, and other uses and methods describe herein. Thus, an aspect of the invention is those nucleic acid sequences that code for cancer-associated polypeptides and polypeptide fragments thereof.

As used herein, a “homolog” to a cancer-associated polypeptide is a polypeptide from a human or other animal that has a high degree of structural similarity to the identified cancer-associated polypeptides. Identification of homologs may be useful in therapeutic drug design or in the production of animal models of cancer. Homologs and alleles of the nucleic acids encoding cancer-associated polypeptides of the invention can be identified by conventional techniques. Identification of human and/or other organism homologs of cancer-associated polypeptides will be familiar to those of skill in the art. In general, nucleic acid hybridization is a suitable method for identification of homologous sequences of another species (e.g., mouse, cow, sheep), which correspond to a known sequence. Standard nucleic acid hybridization procedures can be used to identify related nucleic acid sequences of selected percent identity. For example, one can construct a library of cDNAs reverse transcribed from the mRNA of a selected tissue (e.g., colon, breast, or lung) and use the nucleic acids identified herein to screen the library for related nucleotide sequences. The screening preferably is performed using high-stringency hybridization conditions to identify those sequences that are closely related by sequence identity.

The term “high stringency” as used herein refers to parameters with which the art is familiar. Nucleic acid hybridization parameters may be found in references that compile such methods, e.g. Molecular Cloning: A Laboratory Manual, J. Sambrook, et al., eds., Second Edition, Cold Spring Harbor Laboratory Press, Cold Spring Harbor, N.Y., 1989, or Current Protocols in Molecular Biology, F. M. Ausubel, et al., eds., John Wiley & Sons, Inc., New York. More specifically, high-stringency conditions, as used herein, refers, for example, to hybridization at 65° C. in hybridization buffer (3.5×SSC, 0.02% Ficoll, 0.02% polyvinyl pyrrolidone, 0.02% Bovine Serum Albumin, 2.5 mM NaH₂PO₄ (pH7), 0.5% SDS, 2 mM EDTA). SSC is 0.15M sodium chloride/0.015M sodium citrate, pH7; SDS is sodium dodecyl sulphate; and EDTA is ethylenediaminetetracetic acid. After hybridization, the membrane upon which the DNA is transferred is washed, for example, in 2×SSC at room temperature and then at 0.1-0.5×SSC/0.1×SDS at temperatures up to 68° C.

There are other conditions, reagents, and so forth that can be used, which result in a similar degree of stringency. The skilled artisan will be familiar with such conditions, and thus they are not given here. It will be understood, however, that the skilled artisan will be able to manipulate the conditions in a manner to permit the clear identification of homologs and alleles of cancer-associated polypeptide nucleic acids of the invention (e.g., by using lower stringency conditions). The skilled artisan also is familiar with the methodology for screening cells and libraries for expression of such molecules, which then are routinely isolated, followed by isolation of the pertinent nucleic acid molecule and sequencing.

In general, homologs and alleles typically will share at least 90% nucleotide identity and/or at least 95% amino acid identity to the sequences of cancer-associated nucleic acids and polypeptides, respectively, in some instances will share at least 95% nucleotide identity and/or at least 97% amino acid identity, in other instances will share at least 97% nucleotide identity and/or at least 98% amino acid identity, in other instances will share at least 99% nucleotide identity and/or at least 99% amino acid identity, and in other instances will share at least 99.5% nucleotide identity and/or at least 99.5% amino acid identity. The identity can be calculated using various, publicly available software tools developed by NCBI (Bethesda, Md.) that can be obtained through the internet. Exemplary tools include the BLAST system available from the website of the National Center for Biotechnology Information (NCBI) at the National Institutes of Health. Pairwise and ClustalW alignments (BLOSUM30 matrix setting) as well as Kyte-Doolittle hydropathic analysis can be obtained using the MacVector sequence analysis software (Oxford Molecular Group). Watson-Crick complements of the foregoing nucleic acids also are embraced by the invention. In silico methods can also be used to identify related sequences.

In screening for cancer-associated polypeptide genes, a Southern blot may be performed using the foregoing conditions, together with a detectably labeled probe (e.g. radioactive or chemiluminescent probes). After washing the membrane to which the DNA is finally transferred, the membrane can be placed against X-ray film or a phosphorimager to detect the radioactive or chemiluminescent signal. In screening for the expression of cancer-associated polypeptide nucleic acids, Northern blot hybridizations using the foregoing conditions can be performed on samples taken from cancer patients or subjects suspected of having a condition characterized by abnormal cell proliferation or neoplasia of tissues.

Amplification protocols such as polymerase chain reaction using primers that hybridize to the sequences presented also can be used for detection of the cancer-associated polypeptide genes or expression thereof. Identification of related sequences can also be achieved using polymerase chain reaction (PCR) including RT-PCR, RT-real-time PCR, and other amplification techniques suitable for cloning related nucleic acid sequences. Preferably, PCR primers are selected to amplify portions of a nucleic acid sequence believed to be conserved (e.g., a catalytic domain, a DNA-binding domain, etc.). Again, nucleic acids are preferably amplified from a tissue-specific library (e.g., colon, breast or lung). One also can use expression cloning utilizing the antisera to the polypeptides of the invention to identify nucleic acids that encode related antigenic proteins in humans or other species using the SEREX procedure to screen the appropriate expression libraries. (See: Sahin et al. Proc. Natl. Acad. Sci. USA 92:11810-11813, 1995).

The invention also includes degenerate nucleic acids that include alternative codons to those present in the native materials. For example, serine residues are encoded by the codons TCA, AGT, TCC, TCG, TCT and AGC. Each of the six codons is equivalent for the purposes of encoding a serine residue. Thus, it will be apparent to one of ordinary skill in the art that any of the serine-encoding nucleotide triplets may be employed to direct the protein synthesis apparatus, in vitro or in vivo, to incorporate a serine residue into an elongating cancer-associated polypeptide. Similarly, nucleotide sequence triplets which encode other amino acid residues include, but are not limited to: CCA, CCC, CCG, and CCT (proline codons); CGA, CGC, CGG, CGT, AGA, and AGG (arginine codons); ACA, ACC, ACG, and ACT (threonine codons); AAC and AAT (asparagine codons); and ATA, ATC, and ATT (isoleucine codons). Other amino acid residues may be encoded similarly by multiple nucleotide sequences. Thus, the invention embraces degenerate nucleic acids that differ from the biologically isolated nucleic acids in codon sequence due to the degeneracy of the genetic code.

The invention also provides modified nucleic acid molecules, which include additions, substitutions and deletions of one or more nucleotides (preferably 1-20 nucleotides). In preferred embodiments, these modified nucleic acid molecules and/or the polypeptides they encode retain at least one activity or function of the unmodified nucleic acid molecule and/or the polypeptides, such as antigenicity, receptor binding, etc. In certain embodiments, the modified nucleic acid molecules encode modified polypeptides, preferably polypeptides having conservative amino acid substitutions as are described elsewhere herein. The modified nucleic acid molecules are structurally related to the unmodified nucleic acid molecules and in preferred embodiments are sufficiently structurally related to the unmodified nucleic acid molecules so that the modified and unmodified nucleic acid molecules hybridize under stringent conditions known to one of skill in the art.

For example, modified nucleic acid molecules that encode polypeptides having single amino acid changes can be prepared. Each of these nucleic acid molecules can have one, two or three nucleotide substitutions exclusive of nucleotide changes corresponding to the degeneracy of the genetic code as described herein. Likewise, modified nucleic acid molecules that encode polypeptides having two amino acid changes can be prepared which have, e.g., 2-6 nucleotide changes. Numerous modified nucleic acid molecules like these will be readily envisioned by one of skill in the art, including for example, substitutions of nucleotides in codons encoding amino acids 2 and 3, 2 and 4, 2 and 5, 2 and 6, and so on. In the foregoing example, each combination of two amino acids is included in the set of modified nucleic acid molecules, as well as all nucleotide substitutions which code for the amino acid substitutions. Additional nucleic acid molecules that encode polypeptides having additional substitutions (i.e., 3 or more), additions or deletions (e.g., by introduction of a stop codon or a splice site(s)) also can be prepared and are embraced by the invention as readily envisioned by one of ordinary skill in the art. Any of the foregoing nucleic acids or polypeptides can be tested by routine experimentation for retention of activity or structural relation to the nucleic acids and/or polypeptides disclosed herein.

The invention also provides nucleic acid molecules that encode fragments of cancer-associated polypeptides. Fragments, can be used as probes in Southern and Northern blot assays to identify such nucleic acids, or can be used in amplification assays such as those employing PCR, including, but not limited to RT-PCR and RT-real-time PCR. As known to those skilled in the art, large probes such as 200, 250, 300 or more nucleotides are preferred for certain uses such as Southern and Northern blots, while smaller fragments will be preferred for uses such as PCR. Fragments also can be used to produce fusion proteins for generating antibodies or determining binding of the polypeptide fragments, or for generating immunoassay components. Likewise, fragments can be employed to produce nonfused fragments of the cancer-associated polypeptides, useful, for example, in the preparation of antibodies, and in immunoassays.

The invention also permits the construction of cancer-associated polypeptide gene “knock-out” or “knock-in” cells and/or animals, providing materials for studying certain aspects of cancer and immune system responses to cancer by regulating the expression of cancer-associated polypeptides. For example, a knock-in mouse may be constructed and examined for clinical parallels between the model and a cancer-affected mouse with upregulated expression of a cancer-associated polypeptide, which may be useful to trigger an immune reaction to the polypeptide. Such a cellular or animal model may also be useful for assessing treatment strategies for cancer.

Alternative types of animal models for cancer may be developed based on the invention. Stimulating an immune response to a cancer-associated polypeptide in an animal may provide a model in which to test treatments, and assess the etiology of cancers.

The invention also involves the use of agents such as polypeptides that bind to cancer-associated polypeptides to diagnose cancer. Such binding agents can be used, for example, in screening assays to detect the presence or absence of cancer-associated polypeptides and complexes of cancer-associated polypeptides and their binding partners and can be used in quantitative binding assays to determine levels of expression in biological samples and cells. Such agents also may be used to inhibit the native activity of the cancer-associated polypeptides, for example, by binding to such polypeptides.

The invention, in part, also includes methods of using the molecules of the invention to prevent and/or treat cancer in a subject. For example, cancer treatment may include administering antisense molecules to reduce expression of cancer-associated polypeptides of the invention in the subject. Prevention or treatment methods of the invention may also include administering an effective amount of a molecule, such as an antibody, that specifically binds to a cancer-associated polypeptide in a subject. Such antibodies may inhibit the function of the polypeptide, thereby reducing the cancer phenotype, or the antibodies may include a cytotoxic or radioactive label that kills cells upon binding to the polypeptides of the invention.

The invention also includes in some aspects isolated cancer-associated polypeptides and fragments thereof encoded by the nucleic acid molecules of the invention. Such cancer-associated polypeptides are useful, for example, alone or as fusion proteins to generate antibodies, and as components of an immunoassay or diagnostic assay. Cancer-associated polypeptides can be isolated from biological samples including tissue or cell homogenates, and can also be expressed recombinantly in a variety of prokaryotic and eukaryotic expression systems by constructing an expression vector appropriate to the expression system, introducing the expression vector into the expression system, and isolating the recombinantly expressed protein. Short polypeptides, such as cancer-associated fragments including antigenic peptides also can be synthesized chemically using well-established methods of peptide synthesis.

Fragments of a polypeptide preferably retain a distinct functional capability of the polypeptide. Functional capabilities that can be retained in a fragment of a polypeptide include interaction with antibodies (e.g. antigenic fragments), interaction with other polypeptides or fragments thereof, selective binding of nucleic acids or proteins, and enzymatic activity. One important activity is the ability to provoke in a subject an immune response. As will be recognized by those skilled in the art, the size of the fragment will depend upon factors such as whether the epitope recognized by an antibody is a linear epitope or a conformational epitope. Thus, some antigenic fragments of cancer-associated polypeptides will consist of longer segments while others will consist of shorter segments, (e.g. 5, 6, 7, 8, 9, 10, 11, or 12 or more amino acids long, including each integer up to the full length of the cancer-associated polypeptide). Those skilled in the art are well versed in methods for selecting antigenic fragments of proteins.

The skilled artisan will also realize that conservative amino acid substitutions may be made in cancer-associated polypeptides to provide functionally equivalent variants, or homologs of the foregoing polypeptides, i.e, the variants retain the functional capabilities of the cancer-associated antigen polypeptides. As used herein, a “conservative amino acid substitution” refers to an amino acid substitution that does not alter the relative charge or size characteristics of the protein in which the amino acid substitution is made. Variants can be prepared according to methods for altering polypeptide sequence known to one of ordinary skill in the art such as are found in references that compile such methods, e.g. Molecular Cloning: A Laboratory Manual, J. Sambrook, et al., eds., Second Edition, Cold Spring Harbor Laboratory Press, Cold Spring Harbor, N.Y., 1989, or Current Protocols in Molecular Biology, F. M. Ausubel, et al., eds., John Wiley & Sons, Inc., New York. Exemplary functionally equivalent variants or homologs of the cancer-associated polypeptides include conservative amino acid substitutions of in the amino acid sequences of proteins disclosed herein. Conservative substitutions of amino acids include substitutions made amongst amino acids within the following groups: (a) M, I, L, V; (b) F, Y, W; (c) K, R, H; (d) A, G; (e) S, T; (f) Q, N; and (g) E, D.

For example, upon determining that a peptide is a cancer-associated polypeptide, one can make conservative amino acid substitutions to the amino acid sequence of the peptide, and still have the polypeptide retain its specific antibody-binding characteristics.

Conservative amino-acid substitutions in the amino acid sequence of cancer-associated polypeptides to produce functionally equivalent variants of cancer-associated polypeptides typically are made by alteration of a nucleic acid encoding a cancer-associated polypeptide. Such substitutions can be made by a variety of methods known to one of ordinary skill in the art. For example, amino acid substitutions may be made by PCR-directed mutation, site-directed mutagenesis according to the method of Kunkel (Kunkel, Proc. Nat. Acad. Sci. U.S.A. 82: 488-492, 1985), or by chemical synthesis of a gene encoding a cancer-associated polypeptide. Where amino acid substitutions are made to a small unique fragment of a cancer-associated polypeptide, such as an antigenic epitope recognized by autologous or allogeneic sera or cytolytic T lymphocytes, the substitutions can be made by directly synthesizing the peptide. The activity of functionally equivalent fragments of cancer-associated polypeptides can be tested by cloning the gene encoding the altered cancer-associated polypeptide into a bacterial or mammalian expression vector, introducing the vector into an appropriate host cell, expressing the altered polypeptide, and testing for a functional capability of the cancer-associated polypeptides as disclosed herein. Peptides that are chemically synthesized can be tested directly for function, e.g., for binding to antisera recognizing associated antigens.

The invention as described herein has a number of uses, some of which are described elsewhere herein.

As detailed herein, the foregoing antibodies and other binding molecules may be used for example, to identify tissues expressing a cancer-associated protein. Antibodies also may be coupled to specific diagnostic labeling agents for imaging of cells and tissues that express cancer-associated polypeptides or to therapeutically useful agents according to standard coupling procedures. Diagnostic agents include, but are not limited to, barium sulfate, iocetamic acid, iopanoic acid, ipodate calcium, diatrizoate sodium, diatrizoate meglumine, metrizamide, tyropanoate sodium and radiodiagnostics including positron emitters such as fluorine-18 and carbon-11, gamma emitters such as iodine-123, technitium-99m, iodine-131 and indium-111, nuclides for nuclear magnetic resonance such as fluorine and gadolinium.

The invention also includes methods to monitor the onset, progression, or regression of cancer in a subject by, for example, obtaining cell or tissue samples at sequential times from a subject and assaying such samples for the presence and/or or level of expression of the cancer-associated polypeptides of the invention. A subject may be suspected of having cancer or may be believed not to have cancer and the sample can serve as a baseline level for comparison with subsequent cell or tissue samples from the subject.

Onset of a condition is the initiation of the physiological changes or characteristics associated with the condition in a subject. Such changes may be evidenced by physiological symptoms, or may be clinically asymptomatic. For example, the onset of cancer may be followed by a period during which there may be cancer-associated physiological characteristics in the subject, even though clinical symptoms may not be evident at that time. The progression of a condition follows onset and is the advancement of the physiological characteristics of the condition, which may or may not be marked by an increase in clinical symptoms. In contrast, the regression of a condition is a decrease in physiological characteristics of the condition, perhaps with a parallel reduction in symptoms, and may result from a treatment or may be a natural reversal in the condition.

The presence of a cancer-associated nucleic acid or polypeptide in a cell or tissue sample from a subject that is determined to be at a level above the baseline level for that nucleic acid or polypeptide, is a marker for cancer in the subject. For example, a marker for cancer may be the specific binding of a cancer-associated polypeptide with an antibody. The onset of a cancer condition may be indicated by the appearance of such a marker(s) in a subject's samples where there was no such marker(s) determined previously. For example, if marker(s) for cancer are determined not to be present in a first sample from a subject, the determination that cancer marker(s) are present in a second or subsequent sample from the subject is an indication of the onset of cancer in the subject.

Progression and regression of a cancer condition are indicated by the increase or decrease, respectively, of marker(s) in a subject's samples over time. For example, if marker(s) for cancer are determined to be present in a first sample from a subject and additional marker(s) or more of the initial marker(s) for cancer are determined to be present in a second or subsequent sample from the subject, it indicates the progression of cancer. Regression of cancer is indicated by finding that marker(s) determined to be present in a sample from a subject are not determined to be found, or found at lower amounts in a second or subsequent sample from the subject.

The progression and regression of a cancer condition may also be indicated based on characteristics of the expression of the cancer-associated polypeptides determined in the subject. For example, some cancer-associated polypeptides may be abnormally expressed at specific stages of cancer (e.g. early-stage cancer-associated polypeptides; mid-stage cancer-associated polypeptides; and late-stage cancer-associated polypeptides). Another example, although not intended to be limiting, is that cancer-associated polypeptides may be differentially expressed in primary tumors versus metastases, thereby allowing the stage and/or diagnostic level of the disease to be established, based on the identification of selected cancer-associated polypeptides in a subject sample.

Different types of cancer in a single tissue type may express different cancer-associated polypeptides and the encoding nucleic acid molecules thereof, or may have different spatial or temporal expression patterns. Such variations may allow cancer-specific diagnosis and subsequent treatment tailored to the patient's specific condition. For example, various types of breast cancer [e.g. ductal carcinoma in situ (DCIS), invasive ductal carcinoma (IDC), invasive lobular carcinoma (ILC), invasive pleomorphic lobular carcinoma, inflammatory breast cancer, medullary carcinoma, mucinous carcinoma (also known as colloid carcinoma), and adenocarcinoma], may be differ in that the expression of cancer-associated nucleic acids and polypeptides may be specific for a given type of cancer. For example, in a plurality of subjects with DCIS, a temporal pattern of expression or level of expression of a cancer-associated polypeptide may be identified, that differs from the temporal pattern of expression or level of expression of the same cancer-associated nucleic acid or polypeptide in IDC. These differences in expression, can enable a physician to diagnose the cancer on the basis of differential expression of the cancer-associated polypeptides and the encoding nucleic acid molecules of the invention, and permits specific treatments to be selected and administered on the basis of the differential expression.

The isolation and identification of cancer-associated nucleic acids and polypeptides also permits the artisan to diagnose a disorder characterized by expression of cancer-associated polypeptides, and characterized preferably by an immune response against the cancer-associated polypeptides. In a significant number of tumor-expressed genes and proteins, the increased level of expression in of the cancer-associated polypeptide in a subject with cancer results in a measurable immune response in the subject. The evaluation of this immune response is useful in the diagnosis of cancer in a subject expressing one or more of the cancer-associated polypeptides of the invention.

The methods of the invention related to cancer-associated polypeptide immune responses involve determining the immune response (antibody or cellular) against one or more cancer-associated polypeptides. The immune response can be assayed by any of the various immunoassay methodologies known to one of ordinary skill in the art. For example, the antigenic cancer-associated polypeptides can be used as a target to capture antibodies from a sample, such as a blood sample drawn from a patient in an ELISA assay. Thus, the invention involves in some aspects diagnosing or monitoring cancer in a subject by determining the presence of an immune response to one or more of the cancer-associated polypeptides described herein. In preferred embodiments, this determination is performed by assaying a bodily fluid obtained from the subject, for example blood or lymph node fluid, for the presence of antibodies against one or more cancer-associated polypeptides or the nucleic acid molecules that encode the cancer-associated polypeptides, or for the presence of antibodies against one of the cancer-associated polypeptides as described herein.

Measurement of the immune response against one or more of the cancer-associated polypeptides described herein in a subject over time by sequential determinations is useful to monitor the onset, progression, or regression of cancer including that resulting from a course of treatment. For example, a sample such as blood or lymph node fluid may be obtained from a subject and contacted with a cancer-associated polypeptide of the invention, and binding between an agent in the sample with one of the cancer-associated polypeptides, is an indication of an immune response to the cancer-associated polypeptide. At a second, subsequent time, another sample may be obtained from the subject and similarly tested. The results of the first and second tests (and/or subsequent tests) can be compared as a measure of the onset, regression, or progression of cancer, or, if cancer treatment was undertaken during the interval between obtaining the samples, the effectiveness of the treatment may be evaluated by comparing the results of the two tests.

The stage of cancer in a subject may be determined based on variation in a subject's immune response to cancer-associated polypeptides of the invention. Variability in the immune response to the polypeptides may be used to indicate the stage of cancer in a subject. For example, some cancer-associated polypeptides may trigger an immune response at different stages of the cancer than that triggered by other cancer-associated polypeptides. The cancer-specific diagnoses described herein above may also be based on the variations in immune responses to the different cancer-associated polypeptides.

The invention includes kits for assaying the presence of cancer-associated polypeptides and/or antibodies that specifically bind to cancer-associated polypeptides. An example of a kit may include an antibody or antigen-binding fragment thereof, that binds specifically to a cancer-associated polypeptide. The antibody or antigen-binding fragment thereof, may be applied to a tissue or cell sample from a patient with cancer, suspected of having cancer, or believed to be free of cancer and the sample then processed to assess whether specific binding occurs between the antibody and a polypeptide or other component of the sample.

Another example of such a kit may include the above-mentioned polypeptides bound to a substrate, for example a dipstick, which is dipped into a blood or body fluid sample of a subject. The surface of the substrate may then be processed using procedures well known to those of skill in the art, to assess whether specific binding occurred between the polypeptides and agents (e.g. antibodies) in the subject's sample. For example, procedures may include, but are not limited to, contact with a secondary antibody, or other method that indicates the presence of specific binding.

In addition, the antibody or antigen-binding fragment thereof, may be applied to a body fluid sample, such as blood or lymph node fluid, from a subject, either suspected of having cancer, diagnosed with cancer, or believed to be free of cancer. As will be understood by one of skill in the art, such binding assays may also be performed with a sample or object contacted with an antibody and/or cancer-associated polypeptide that is in solution, for example in a 96-well plate or applied directly to an object surface.

Another example of a kit of the invention, is a kit that provides components necessary to determine the level of expression of one or more cancer-associated nucleic acid molecules of the invention. Such components may include primers useful for amplification of one or more cancer-associated nucleic acid molecules and/or other chemicals for PCR amplification.

Another example of a kit of the invention, is a kit that provides components necessary to determine the level of expression of one or more cancer-associated nucleic acid molecules of the invention using a method of hybridization.

The foregoing kits can include instructions or other printed material on how to use the various components of the kits for diagnostic purposes.

The cancer-associated polypeptides of the invention, including fragments thereof, can also be used to screen peptide libraries, including phage display libraries, to identify and select peptide binding partners of the cancer-associated polypeptides of the invention. Such molecules can be used, as described, for screening assays, for purification protocols, for interfering directly with the functioning of cancer-associated polypeptides and for other purposes that will be apparent to those of ordinary skill in the art. For example, isolated cancer-associated polypeptides can be attached to a substrate (e.g., chromatographic media, such as polystyrene beads, or a filter), and then a solution suspected of containing the binding partner may be applied to the substrate. If a binding partner that can interact with cancer-associated polypeptides is present in the solution, then it will bind to the substrate-bound cancer-associated polypeptide. The binding partner then may be isolated.

The invention, therefore, embraces polypeptide binding agents which, for example, can be antibodies or fragments of antibodies having the ability to selectively bind to cancer-associated polypeptides. Antibodies include polyclonal and monoclonal antibodies, prepared according to conventional methodology.

Significantly, as is well-known in the art, only a small portion of an antibody molecule, the paratope, is involved in the binding of the antibody to its epitope (see, in general, Clark, W. R. (1986) The Experimental Foundations of Modern Immunology Wiley & Sons, Inc., New York; Roitt, I. (1991) Essential Immunology, 7th Ed., Blackwell Scientific Publications, Oxford). The pFc′ and Fc regions, for example, are effectors of the complement cascade but are not involved in antigen binding. An antibody from which the pFc′ region has been enzymatically cleaved, or which has been produced without the pFc′ region, designated an F(ab′)₂ fragment, retains both of the antigen binding sites of an intact antibody. Similarly, an antibody from which the Fe region has been enzymatically cleaved, or which has been produced without the Fc region, designated an Fab fragment, retains one of the antigen binding sites of an intact antibody molecule. Proceeding further, Fab fragments consist of a covalently bound antibody light chain and a portion of the antibody heavy chain denoted Fd. The Fd fragments are the major determinant of antibody specificity (a single Fd fragment may be associated with up to ten different light chains without altering antibody specificity) and Fd fragments retain epitope-binding ability in isolation.

Within the antigen-binding portion of an antibody, as is well known in the art, there are complementarity determining regions (CDRs), which directly interact with the epitope of the antigen, and framework regions (FRs), which maintain the tertiary structure of the paratope (see, in general, Clark, 1986; Roitt, 1991). In both the heavy chain Fd fragment and the light chain of IgG immunoglobulins, there are four framework regions (FR1 through FR4) separated respectively by three complementarity determining regions (CDR1 through CDR3). The CDRs, and in particular the CDR3 regions, and more particularly the heavy chain CDR3, are largely responsible for antibody specificity.

It is now well established in the art that the non-CDR regions of a mammalian antibody may be replaced with similar regions of conspecific or heterospecific antibodies while retaining the epitopic specificity of the original antibody. This is most clearly manifested in the development and use of “humanized” antibodies in which non-human CDRs are covalently joined to human FR and/or Fc/pFc′ regions to produce a functional antibody. See, e.g., U.S. Pat. Nos. 4,816,567, 5,225,539, 5,585,089, 5,693,762 and 5,859,205.

Fully human monoclonal antibodies also can be prepared by immunizing mice transgenic for large portions of human immunoglobulin heavy and light chain loci. Following immunization of these mice (e.g., XenoMouse (Abgenix), HuMAb mice (Medarex/GenPharm)), monoclonal antibodies can be prepared according to standard hybridoma technology. These monoclonal antibodies will have human immunoglobulin amino acid sequences and therefore will not provoke human anti-mouse antibody (HAMA) responses when administered to humans.

Thus, as will be apparent to one of ordinary skill in the art, the present invention also provides for F(ab′)₂, Fab, Fv and Fd fragments; chimeric antibodies in which the Fc and/or FR and/or CDR1 and/or CDR2 and/or light chain CDR3 regions have been replaced by homologous human or non-human sequences; chimeric F(ab′)₂ fragment antibodies in which the FR and/or CDR1 and/or CDR2 and/or light chain CDR3 regions have been replaced by homologous human or non-human sequences; chimeric Fab fragment antibodies in which the FR and/or CDR1 and/or CDR2 and/or light chain CDR3 regions have been replaced by homologous human or non-human sequences; and chimeric Fd fragment antibodies in which the FR and/or CDR1 and/or CDR2 regions have been replaced by homologous human or non-human sequences. The present invention also includes so-called single chain antibodies.

Thus, the invention involves polypeptides of numerous size and type that bind specifically to cancer-associated polypeptides, and complexes of both cancer-associated polypeptides and their binding partners. These polypeptides may be derived also from sources other than antibody technology. For example, such polypeptide binding agents can be provided by degenerate peptide libraries which can be readily prepared in solution, in immobilized form or as phage display libraries. Combinatorial libraries also can be synthesized of peptides containing one or more amino acids. Libraries further can be synthesized of peptoids and non-peptide synthetic moieties.

Phage display can be particularly effective in identifying binding peptides useful according to the invention. Briefly, one prepares a phage library (using e.g. m13, fd, or lambda phage), displaying inserts from 4 to about 80 amino acid residues using conventional procedures. The inserts may represent, for example, a completely degenerate or biased array. One then can select phage-bearing inserts which bind to the cancer-associated polypeptide. This process can be repeated through several cycles of reselection of phage that bind to the cancer-associated polypeptide. Repeated rounds lead to enrichment of phage bearing particular sequences. DNA sequence analysis can be conducted to identify the sequences of the expressed polypeptides. The minimal linear portion of the sequence that binds to the cancer-associated polypeptide can be determined. One can repeat the procedure using a biased library containing inserts containing part or all of the minimal linear portion plus one or more additional degenerate residues upstream or downstream thereof. Yeast two-hybrid screening methods also may be used to identify polypeptides that bind to the cancer-associated polypeptides.

Optionally, an antibody can be linked to one or more detectable markers (as described herein), antitumor agents, or immunomodulators. Antitumor agents can include cytotoxic agents and agents that act on tumor neovasculature. Detectable markers include, for example, radioactive or fluorescent markers. Cytotoxic agents include cytotoxic radionuclides, chemical toxins and protein toxins.

The cytotoxic radionuclide or radiotherapeutic isotope may be an alpha-emitting isotope such as ²²⁵Ac, ²¹¹At, ²¹²Bi, or ²¹³Bi. Alternatively, the cytotoxic radionuclide may be a beta-emitting isotope such as ¹⁸⁶Rh, ¹⁸⁸Rh, ⁹⁰Y, ¹³¹I or ⁶⁷Cu. Further, the cytotoxic radionuclide may emit Auger and low-energy electrons such as the isotopes ¹²⁵I, ¹²³I, or ⁷⁷Br.

Suitable chemical toxins or chemotherapeutic agents include members of the enediyne family of molecules, such as chalicheamicin and esperamicin. Chemical toxins can also be taken from the group consisting of methotrexate, doxorubicin, melphalan, chlorambucil, ARA-C, vindesine, mitomycin C, cis-platinum, etoposide, bleomycin and 5-fluorouaracil. Agents that act on the tumor neovasculature can include tubulin-binding agents such as combrestatin A4 (Griggs et al., Lancet Oncol. 2:82, 2001) and angiostatin and endostatin (reviewed in Rosen, Oncologist 5:20, 2000, incorporated by reference herein). Other chemotherapeutic agents are known to those skilled in the art.

The invention includes methods of preventing and/or treating cancer in a subject. Such methods include administering a pharmaceutical agent or compound of the invention in an amount effective to prevent or treat cancer in a subject. For example, a pharmaceutical compound that includes an antibody, as described herein, can be administered to prevent or treat cancer in a subject. The effectiveness of treatment or prevention methods of the invention can be determined using standard diagnostic methods described herein.

When administered, the pharmaceutical compositions of the present invention are administered in pharmaceutically acceptable preparations. Such preparations may routinely contain pharmaceutically acceptable concentrations of salt, buffering agents, preservatives, compatible carriers, and optionally other therapeutic agents.

The term “pharmaceutically acceptable” means a non-toxic material that does not interfere with the effectiveness of the biological activity of the active ingredients. The characteristics of the carrier will depend on the route of administration.

The pharmaceutical compositions of the invention can be administered by any conventional route, including injection or by gradual infusion over time. The administration may, for example, be oral, intravenous, intraperitoneal, intramuscular, intranasal, intracavity, subcutaneous, intradermal, or transdermal.

Preparations for parenteral administration include sterile aqueous or non-aqueous solutions, suspensions, and emulsions. Examples of non-aqueous solvents are propylene glycol, polyethylene glycol, vegetable oils such as olive oil, and injectable organic esters such as ethyl oleate. Aqueous carriers include water, alcoholic/aqueous solutions, emulsions or suspensions, including saline and buffered media. Parenteral vehicles include sodium chloride solution, Ringer's dextrose, dextrose and sodium chloride, lactated Ringer's or fixed oils. Intravenous vehicles include fluid and nutrient replenishers, electrolyte replenishers (such as those based on Ringer's dextrose), and the like. Preservatives and other additives may also be present such as, for example, antimicrobials, anti-oxidants, chelating agents, and inert gases and the like.

The pharmaceutical compound or agent dosage may be adjusted by a physician or veterinarian, particularly in the event of any complication. A therapeutically effective amount typically varies from 0.01 mg/kg to about 1000 mg/kg, preferably from about 0.1 mg/kg to about 200 mg/kg, and most preferably from about 0.2 mg/kg to about 20 mg/kg, in one or more dose administrations for one or more days.

The absolute amount of a pharmaceutical compound that is administered will depend upon a variety of factors, including the material selected for administration, whether the administration is in single or multiple doses, and individual patient parameters including age, physical condition, size, weight, and the stage of the disease. These factors are well known to those of ordinary skill in the art and can be addressed with no more than routine experimentation.

The determination of whether treatment in a subject is effective, and/or whether the amount administered is a therapeutically effective amount can be done using routine methods known those of ordinary skill in the art. For example, diagnostic tests known to those of ordinary skill in the art or as described herein, may be used to assess the cancer status of a subject and evaluate the effectiveness of a pharmaceutical compound or agent that has been administered to the subject. A first determination of cancer may be obtained using one of the methods described herein (or other methods known in the art), and a subsequent determination of the presence of cancer in a subject may be done. A comparison of the presence of cancer, for example by determining the expression level of a polypeptide or nucleic acid molecule of the invention, may be used to assess the effectiveness of administration of a pharmaceutical compound or agent of the invention as a prophylactic or a treatment of the cancer. A level of expression of the nucleic acid or polypeptide molecules of the invention that is above the baseline control level of expression for that tissue may be an indication of a need for treatment intervention by administering a pharmaceutical agent described herein to prevent or treat cancer.

The pharmaceutical agents of the invention may be administered alone, in combination with each other, and/or in combination with other anti-cancer drug therapies and/or treatments. These therapies and/or treatments may include, but are not limited to: surgical intervention, chemotherapy, radiotherapy, and adjuvant systemic therapies.

The invention also provides a pharmaceutical kit comprising one or more containers comprising one or more of the pharmaceutical compounds or agents of the invention. The kit may also include instructions for the use of the one or more pharmaceutical compounds or agents of the invention for the treatment of cancer.

The invention further includes nucleic acid or protein microarrays having cancer-associated peptides or nucleic acids encoding such polypeptides. In this aspect of the invention, standard techniques of microarray technology are utilized to assess expression of the cancer-associated nucleic acids or polypeptides and/or identify biological constituents that bind such nucleic acids or polypeptides. The constituents of biological samples include antibodies, lymphocytes (particularly T lymphocytes), and the like. Protein microarray technology, which is also known by other names including: protein chip technology and solid-phase protein array technology, is well known to those of ordinary skill in the art and is based on, but not limited to, obtaining an array of identified peptides or proteins on a fixed substrate, binding target molecules or biological constituents to the peptides, and evaluating such binding. See, e.g., G. MacBeath and S. L. Schreiber, “Printing Proteins as Microarrays for High-Throughput Function Determination,” Science 289(5485):1760-1763, 2000. Nucleic acid arrays, particularly arrays that bind cancer-associated peptides, also can be used for diagnostic applications, such as for identifying subjects that have a condition characterized by cancer-associated polypeptide expression.

Microarray substrates include but are not limited to glass, silica, aluminosilicates, borosilicates, metal oxides such as alumina and nickel oxide, various clays, nitrocellulose, or nylon. The microarray substrates may be coated with a compound to enhance synthesis of a probe (peptide or nucleic acid) on the substrate. Coupling agents or groups on the substrate can be used to covalently link the first nucleotide or amino acid to the substrate. A variety of coupling agents or groups are known to those of skill in the art. Peptide or nucleic acid probes thus can be synthesized directly on the substrate in a predetermined grid. Alternatively, peptide or nucleic acid probes can be spotted on the substrate, and in such cases the substrate may be coated with a compound to enhance binding of the probe to the substrate. In these embodiments, presynthesized probes are applied to the substrate in a precise, predetermined volume and grid pattern, preferably utilizing a computer-controlled robot to apply probe to the substrate in a contact-printing manner or in a non-contact manner such as ink jet or piezo-electric delivery. Probes may be covalently linked to the substrate.

Targets are peptides or proteins and may be natural or synthetic. The tissue may be obtained from a subject or may be grown in culture (e.g. from a cell line).

In some embodiments of the invention, one or more control peptide or protein molecules are attached to the substrate. Preferably, control peptide or protein molecules allow determination of factors such as peptide or protein quality and binding characteristics, reagent quality and effectiveness, hybridization success, and analysis thresholds and success.

Nucleic acid microarray technology, which is also known by other names including: DNA chip technology, gene chip technology, and solid-phase nucleic acid array technology, is well known to those of ordinary skill in the art and is based on, but not limited to, obtaining an array of identified nucleic acid probes on a fixed substrate, labeling target molecules with reporter molecules (e.g., radioactive, chemiluminescent, or fluorescent tags such as fluorescein, Cye3-dUTP, or Cye5-dUTP), hybridizing target nucleic acids to the probes, and evaluating target-probe hybridization. A probe with a nucleic acid sequence that perfectly matches the target sequence will, in general, result in detection of a stronger reporter-molecule signal than will probes with less perfect matches. Many components and techniques utilized in nucleic acid microarray technology are presented in The Chipping Forecast, Nature Genetics, Vol. 21, January 1999, the entire contents of which is incorporated by reference herein.

According to the present invention, nucleic acid microarray substrates may include but are not limited to glass, silica, aluminosilicates, borosilicates, metal oxides such as alumina and nickel oxide, various clays, nitrocellulose, or nylon. According to the invention, probes are selected from the group of nucleic acids including, but not limited to: DNA, genomic DNA, cDNA, and oligonucleotides; and may be natural or synthetic. Oligonucleotide probes preferably are 20 to 25-mer oligonucleotides and DNA/cDNA probes preferably are 500 to 5000 bases in length, although other lengths may be used. Appropriate probe length may be determined by one of ordinary skill in the art by following art-known procedures. In one embodiment, preferred probes are sets of more than two of the cancer-associated polypeptide nucleic acid molecules set forth herein, or one of the novel cancer-associated polypeptide nucleic acid molecules as described herein. Probes may be purified to remove contaminants using standard methods known to those of ordinary skill in the art such as gel filtration or precipitation.

In one embodiment, the microarray substrate may be coated with a compound to enhance synthesis of the probe on the substrate. Such compounds include, but are not limited to, oligoethylene glycols. In another embodiment, coupling agents or groups on the substrate can be used to covalently link the first nucleotide or olignucleotide to the substrate. These agents or groups may include, for example, amino, hydroxy, bromo, and carboxy groups. These reactive groups are preferably attached to the substrate through a hydrocarbyl radical such as an alkylene or phenylene divalent radical, one valence position occupied by the chain bonding and the remaining attached to the reactive groups. These hydrocarbyl groups may contain up to about ten carbon atoms, preferably up to about six carbon atoms. Alkylene radicals are usually preferred containing two to four carbon atoms in the principal chain. These and additional details of the process are disclosed, for example, in U.S. Pat. No. 4,458,066, which is incorporated by reference in its entirety.

In one embodiment, probes are synthesized directly on the substrate in a predetermined grid pattern using methods such as light-directed chemical synthesis, photochemical deprotection, or delivery of nucleotide precursors to the substrate and subsequent probe production.

In another embodiment, the substrate may be coated with a compound to enhance binding of the probe to the substrate. Such compounds include, but are not limited to: polylysine, amino silanes, amino-reactive silanes (Chipping Forecast, 1999) or chromium. In this embodiment, presynthesized probes are applied to the substrate in a precise, predetermined volume and grid pattern, utilizing a computer-controlled robot to apply probe to the substrate in a contact-printing manner or in a non-contact manner such as ink jet or piezo-electric delivery. Probes may be covalently linked to the substrate with methods that include, but are not limited to, UV-irradiation. In another embodiment probes are linked to the substrate with heat.

Targets for microarrays are nucleic acids selected from the group, including but not limited to: DNA, genomic DNA, cDNA, RNA, mRNA and may be natural or synthetic. In all embodiments, nucleic acid target molecules from human tissue are preferred. The tissue may be obtained from a subject or may be grown in culture (e.g. from a cell line).

In embodiments of the invention one or more control nucleic acid molecules are attached to the substrate. Preferably, control nucleic acid molecules allow determination of factors such as nucleic acid quality and binding characteristics, reagent quality and effectiveness, hybridization success, and analysis thresholds and success. Control nucleic acids may include but are not limited to expression products of genes such as housekeeping genes or fragments thereof.

In some embodiments, one or more control nucleic acid molecules are attached to the substrate. Preferably, control nucleic acid molecules allow determination of factors such as binding characteristics, reagent quality and effectiveness, hybridization success, and analysis thresholds and success.

### EXAMPLES

Background

An important class of tumor-specific antigens is encoded by genes, such as MAGE, LAGE, BAGE, GAGE and NY-ESO-1, which are normally expressed exclusively in male germ-line cells but become aberrantly activated in various tumors. A certain number of these genes appear to be expressed only in spermatogonia but not in other stages of spermatogenesis. We therefore hypothesized that genes with spermatogonia-specific expression might be prone to frequent activation in tumoral tissues. Here, we tested the tumoral expression of a panel of 14 genes whose spermatogonia-specific expression has been recently described by Wang et al., (2001) Nature Genetics 27:422-426.

Using RT-PCR, we analyzed the expression of these genes in a series of tumor cell lines and normal tissue samples. Five genes, namely NXF2, TAF2Q, FTHL17, TEX15, and TDRD1 presented a significant PCR signal in at least one tumor cell line, whereas their expression in normal somatic tissues was either absent or very weak. Weak expressions in normal somatic tissues were quantified by real-time PCR, and were found to represent only 0.2 to 1.8% of the expression level in testis. These five genes were selected for further RT-PCR analysis in a large number of tumoral tissue samples of various histological types. All five genes were found to be expressed in a significant fraction of tumor samples. Real-time PCR experiments showed that their levels of expression in tumor samples were significantly higher than the background expression levels observed in some normal somatic tissues. Due to their cancer/spermatogonia specific expression, NXF2, TAF2Q, FTHL17, TEX15 and TDRD1 genes are likely to produce tumor-specific antigens.

### Example 1

### NXF2 Nuclear RNA Export Factor 2

Introduction

NXF2 is mapped on the X chromosome. It encodes a protein involved in nuclear RNA export. NXF2 is listed in GenBank under Accession number: AF285596. The expression of NXF2 was tested in 21 tumoral cell lines by RT-PCR. NXF2 expression was detected in 5 cell lines, including two melanomas cell lines, two lung cell lines and one sarcoma cell line.

Methods

RT-PCR Conditions

Reverse transcription was performed on 2 μg of total RNA with 200 units of M-MLV reverse transcriptase (GibcoBRL-Life Technologies, Rockville, Md.). cDNA corresponding to 50 ng of reverse-transcribed RNA was then amplified by PCR with primers NXF2_sens 5′-ctattcccttcgacccca-3′ (SEQ ID NO: 11) and NXF2_as 5′-ctctttgggtggttatgtcac-3′ (SEQ ID NO: 12), and with 0.625 units of TaKaRa Taq polymerase. Cycling conditions were 30 cycles (30 sec at 94° C.; 30 sec at 64° C.; 1 min at 72° C.) and 10 min at 72° C. for final extension. No product was amplified from human genomic DNA under these conditions. Specific amplification was confirmed by the sequencing of the testis PCR product.

RT-Real-Time PCR Conditions

The quality and the concentration of RNA samples were checked in an ethidium bromide staining gel. The relative amounts of RNA were corrected according to the intensity of the RNA 28S band measured with a Kodak Digital Science Image Station 440CF (Eastman Kodak Co., Rochester, N.Y.). 0.75 μg of total RNA samples were reverse transcribed with 200 units of M-MLV reverse transcriptase (GibcoBRL-Life Technologies). The quantitation of NXF2 mRNA levels was carried out by a real-time fluorescence detection method, using the ABI Prism 7700 sequence detector (Applied Biosystems, Foster City, Calif.). cDNA corresponding to 1.875 ng of reverse transcribed RNA was amplified by real-time-PCR with 0.625 units Hot GoldStar enzyme and 0.25 units Uracyl-N-glycosylase (Eurogentec SA, Seraing, Belgium). The reaction was performed using the NXF2 primers described above and 5′FAM-cactaaggacccctacctgaag-3′TAMRA (SEQ ID NO: 13), as a probe with carboxyfluorescein (FAM) and carboxytetramethylrhodamine (TAMRA). The thermal conditions were 2 min at 50° C., 10 min at 95° C., 50 cycles (15 sec at 95° C.; 2 min at 64° C.).

Results

To ensure that NXF2 was strictly testis-specific, the NXF2 expression in normal tissues was analyzed by RT-PCR (Table 3). No signal was detected in any normal tissue tested, other than testis, except in trachea where a very weak signal was detected. The level of NXF2 expression in trachea was quantified by RT-real-time PCR and it was only 1% of the expression level found in testis.

RT-PCR was used to screen a large number of tumoral samples of various histological origins (Table 4). Overall, expression of NXF2 appeared to be reactivated in 10% of all tested tumors. Its expression frequency reached 27% (3/11) in sarcomas, 19% (4/21) in bladder carcinomas, 15% (4/26) in lung carcinomas, and 11% (2/18) in colorectal carcinomas.

Finally, RT-real-time-PCR was used to quantify NXF2 expression level in some tumoral samples. Table 5 shows that NXF2 was expressed at high levels in some tumors (up to 342% of the testis expression level).

TABLE 3
NXF2 expression in normal tissues

| | | | RT-real-time-PCR analysis |
|---|---|---|---|
| | | | \overline{Percent of NXF2 level expression in} |
| Tissue sample | Patient code | RT-PCR analysis | normal tissues in reference to testis |
| Testis | LB 882 | +++ | / |
| Testis | CLO 26 | +++ | 100% |
| Adrenal gland | 808 LB 535 | − | / |
| adrenal gland | CLO 32 | − | / |
| Bladder | 1598 HM81 915-1 | − | / |
| bone marrow | 3109 LB 1788 | − | / |
| bone marrow | CLO 21 | − | / |
| Brain | 878 JNO 9 | − | / |
| Brain | CLO 14 | − | / |
| Breast | 408 LB 223 | − | / |
| Cerebellum | 642 CLO 6 64035 | − | / |
| Colon | 428 LB 298 | − | / |
| Colon | CLO 20 | − | / |
| Epididyme | 1307 LB 881 | − | / |
| Heart | 819 CLO 3 64025 | − | / |
| Heart | CLO 15 | − | / |
| Kidney | 683 BA 4 | − | / |
| Kidney | CLO 16 | − | / |
| Liver | CLO 17 | − | / |
| Liver | 1765 CLO 10 64022-1 | − | / |
| Lung | CLO 18 | − | / |
| Lymphocytes | 3151 LB41 | − | / |
| (PBLs) | | | |
| Muscle | 2967 CLO 12 64033-1 | − | / |
| Muscle | CLO 25 | − | / |
| Ovary | 737 CLO 7 64036 | − | / |
| Placenta | CLO 31 | − | / |
| Prostate | 643 CLO 9 64038 | − | / |
| Prostate | CLO 24 | − | / |
| Skin | 831 LB 149 | − | / |
| Spleen | CLO 22 | − | / |
| Thymus | 308 LB646 | − | / |
| Thymus | CLO 23 | − | / |
| Trachea | CLO 19 | + | 1% |
| Uterus | 2966 CLO 11 64029-1 | − | / |
| Uterus | CLO 27 | − | / |
| Uterus | 1744 LB 1031 | − | / |

+++, + and − refer to the PCR signal intensity
/: not tested

TABLE 4
RT-PCR analysis of NXF2 expression in tumor samples

| | Number of positive tumoral samples/ | |
|---|---|---|
| Tumoral samples | Number of tumoral samples tested | |
| Sarcomas | 3/11 | (27%) |
| Bladder carcinomas | 4/21 | (19%) |
| Lung carcinomas NSCLC | 4/26 | (15%) |
| Colorectal carcinomas | 2/18 | (11%) |
| Thyroid tumors | 1/5 | |
| Esophageal carcinomas | 1/8 | |
| Prostate tumors | 1/7 | |
| Head and neck epidermoid | 1/19 | |
| carcinomas | | |
| Cutaneous melanomas | 1/18 | |
| Neuroblastomas | 1/1 | |
| Uveal melanomas | 0/3 | |
| Brain tumors | 0/7 | |
| Uterine tumors | 0/5 | |
| Myelomas | 0/1 | |
| Mesotheliomas | 0/2 | |
| Leukemias | 0/18 | |
| Renal tumors | 0/16 | |
| Breast carcinomas | 0/12 | |
| | 19/198 | (10%) |

TABLE 5
Quantification of NXF2 level expression in tumoral samples

| | | RT-real-time-PCR analysis |
|---|---|---|
| | | \overline{Percent of NXF2 level expression in} |
| Tissue sample | Patient code | tumoral tissues referred to testis. |
| Normal testis | HM 31 1A5 | 100 |
| Bladder | HM 50 281 | 16 |
| carcinoma | | |
| Bladder | HM 14 407 | 2 |
| carcinoma | | |
| Colorectal | LB 728 | 106 |
| carcinoma | | |
| Colorectal | LB 989 | 9 |
| carcinoma | | |
| Cutaneous | CPVB 50.I | 6 |
| melanoma | | |
| Esophagial | LB 1472 | 342 |
| carcinoma | | |
| Head and neck | LB 1017 | 24 |
| carcinoma | | |
| Lung carcinoma | LB 1135 | 33 |
| NSCLC | | |
| Lung carinoma | LB 1136 | 23 |
| NSCLC | | |
| Lung carinoma | LB 1061 | 21 |
| NSCLC | | |
| Lung carinoma | LB 1080 | 6 |
| NSCLC | | |
| Neuroblastoma | LB 837 | 6 |
| Prostate tumor | LB 505 | 6 |
| Sarcoma | LB 766 | 22 |
| Sarcoma | LB 1443 | 38 |
| Thyroid tumor | LB 1163 | 18 |

### Example 2

### TAF2Q TATA Box-Binding Protein-Associated Factor Q

Introduction

TAF2Q is mapped on the X chromosome. It encodes a RNA polymerase II TBP-associated factor. TAF2Q is listed in GenBank under Accession number: AF285595. The expression of TAF2Q was tested in 22 tumoral cell lines by RT-PCR. TAF2Q expression was detected in 5 cell lines, including two melanomas cell lines, two lung cell lines and one bladder carcinoma cell line.

Methods

RT-PCR Conditions

Reverse transcription was performed on 2 μg of total RNA with 200 units of M-MLV reverse transcriptase (GibcoBRL-Life Technologies). cDNA corresponding to 50 ng of reverse-transcribed RNA was then amplified by PCR with primers TAF2Q_sens 5′-gaagtaaagagactgctgcgt-3′ (SEQ ID NO: 14) and TAF2Q_as 5′-cgagtggggtctttaagtcta-3′ (SEQ ID NO: 15), and with 0.625 units of TaKaRa Taq polymerase. Cycling conditions were 30 cycles (30 sec at 94° C.; 30 sec at 60° C.; 1 min at 72° C.) and 10 min at 72° C. for final extension. No product was amplified from human genomic DNA under these conditions. Specific amplification was confirmed by the sequencing of the testis PCR product.

RT-Real-Time PCR Conditions

The quality and the concentration of RNA samples were checked in an ethidium bromide staining gel. The relative amounts of RNA were corrected according to the intensity of the RNA 28S band measured with a Kodak Digital Science Image Station 440CF (Eastman Kodak Co.). 0.75 μg of total RNA samples were reverse transcribed with 200 units of M-MLV reverse transcriptase (GibcoBRL-Life Technologies). The quantitation of TAF2Q mRNA levels was carried out by a real-time fluorescence detection method, using the ABI Prism 7700 sequence detector (Applied Biosystems). cDNA corresponding to 1.25 ng of reverse transcribed RNA was amplified by real-time-PCR with 0.625 units Hot GoldStar enzyme and 0.25 units Uracyl-N-glycosylase (Eurogentec SA). The reaction was performed using the TAF2Q primers described above and 5′FAM-agaaagtcaaggctccatcccag-3′TAMRA (SEQ ID NO: 16) as a probe. The thermal conditions were 2 min at 50° C., 10 min at 95° C., 50 cycles (15 sec at 95° C.; 2 min at 60° C.).

Results

To ensure that TAF2Q was strictly testis-specific, the TAF2Q expression in normal tissues was analyzed by RT-PCR (Table 6). No signal was detected in any normal tissue tested, other than testis.

RT-PCR was used to screen a large number of tumoral samples of various histological origins (Table 7). Overall, expression of TAF2Q appeared to be reactivated in 8% of all tested tumors. Its expression frequency reached 28% (4/14) in cutaneous melanomas and 25% (2/8) in sarcomas.

Finally, RT-real-time-PCR was used to quantify the TAF2Q expression level in some tumoral samples. Table 8. shows that TAF2Q was expressed at significant levels in some tumors (up to 28% of the testis expression level).

TABLE 6
TAF2Q expression in normal tissues

| Tissue sample | Patient code | RT-PCR analysis |
|---|---|---|
| Testis | LB 882 | +++ |
| Testis | CLO 26 | +++ |
| Adrenal gland | CLO 32 | − |
| Bone marrow | CLO 21 | − |
| Brain | CLO 14 | − |
| Colon | CLO 20 | − |
| Heart | CLO 15 | − |
| Kidney | CLO 16 | − |
| Liver | CLO 17 | − |
| Lung | LB 195 | − |
| Lung | CLO 18 | − |
| Melanocytes | LB 1878 | − |
| Muscle | CLO 25 | − |
| Ovary | LB 2040 | − |
| Placenta | CLO 31 | − |
| Prostate | CLO 24 | − |
| Spleen | CLO 22 | − |
| Thymus | CLO 23 | − |
| Trachea | CLO 19 | − |
| Uterus | CLO 27 | − |

+++ and − refer to the PCR signal intensity

TABLE 7
RT-PCR analysis of TAF2Q expression in tumor samples

| | Number of positive tumoral samples/ | |
|---|---|---|
| Tumoral samples | Number of tumoral samples tested | |
| Cutaneous melanomas | 4/14 | (28%) |
| Sarcomas | 2/8 | (25%) |
| Lung carcinomas NSCL C | 1/11 | |
| Bladder carcinomas | 1/10 | |
| Head and neck | 1/10 | |
| epidermoid carcinoma | | |
| Renal tumors | 0/9 | |
| Breast carcinomas | 0/7 | |
| Prostate tumors | 0/6 | |
| Esophagial carcinomas | 0/5 | |
| Leukemias | 0/5 | |
| Uterine tumors | 0/5 | |
| Brain tumors | 0/4 | |
| Thyroid tumors | 0/4 | |
| Myelomas | 0/3 | |
| Neuroblastomas | 0/2 | |
| Colorectal carcinomas | 0/11 | |
| Mesotheliomas | 0/1 | |
| Total | 9/115 | (8%) |

TABLE 8
Quantification of TAF2Q level expression in tumoral sample

| | | RT-real-time-PCR analysis |
|---|---|---|
| | | \overline{Percent of NXF2 level expression in} |
| Tissue sample | Patient code | tumoral tissues referred to testis. |
| Normal testis | HM 31 1A5 | 100 |
| Bladder | HM 14 407 | 1 |
| carcinoma | | |
| Cutaneous | LB 1610 | 14 |
| melanoma | | |
| Cutaneous | LB 1616 | 1 |
| melanoma | | |
| Cutaneous | CPVB 30 | 28 |
| melanoma | | |
| Cutaneous | CPVB 50.II | 6 |
| melanoma | | |
| Head and neck | BB 61 | 12 |
| carcinoma | | |
| Lung carcinoma | LB 1135 | 2 |
| Sarcoma | LB 1443 | 15 |
| Sarcoma | LB 766 | 1 |

### Example 3

### FTHL17 Ferritin Heavy Polypeptide-like 17

Introduction

FTHL17 is located on the X chromosome. It codes for a protein potentially involved in iron metabolism. FTHL17 is listed in GenBank under Accession number: AF285592. The expression of FTHL17 was tested in 20 tumoral cell lines by RT-PCR. FTHL17 expression was detected in a sarcoma cell line.

Methods

RT-PCR Conditions

Reverse transcription was performed on 2 μg of total RNA with 200 units of M-MLV reverse transcriptase (GibcoBRL-Life Technologies). cDNA corresponding to 50 ng of reverse-transcribed RNA was then amplified by PCR with PrimerA 5′-tggccctggagaacttctt-3′ (SEQ ID NO: 17) and PrimerB 5′-atggtcttgacttgctcgtg-3′ (SEQ ID NO: 18) described by Wang et al. (Accession number G65765), and with 0.625 units of TaKaRa Taq polymerase. Cycling conditions were 30 cycles (30 sec at 94° C.; 30 sec at 60° C.; 1 min at 72° C.) and 10 min at 72° C. for final extension. Because FTHL17 PCR primers are located within the same exon, positive samples were tested for FTHL17 amplification from non-reverse RNA to eliminate false positives resulting from the presence of contaminating genomic DNA. Specific amplification was confirmed by the sequencing of the testis PCR product.

RT-Real-Time PCR Conditions

The quality and the concentration of RNA samples were checked in an ethidium bromide staining gel. The relative amounts of RNA were corrected according to the intensity of the RNA 28S band measured with a Kodak Digital Science Image Station 440CF (Eastman Kodak, Co.). 0.75 μg of total RNA samples were reverse transcribed with 200 units of M-MLV reverse transcriptase (GibcoBRL-Life Technologies). The quantitation of FTHL17 mRNA levels was carried out by a real-time fluorescence detection method, using the ABI Prism 7700 sequence detector (Applied Biosystems). cDNA corresponding to 1.25 ng of reverse transcribed RNA was amplified by real-time-PCR with 0.625 units Hot GoldStar enzyme and 0.25 units Uracyl-N-glycosylase (Eurogentec SA). The reaction was performed using the FTHL17 primers described above and 5′FAM-tggccacatctgccttcacg-3′TAMRA (SEQ ID NO: 19) as a probe. The thermal conditions were 2 min at 50° C., 10 min at 95° C., 50 cycles (15 sec at 95° C.; 1 min at 60° C.).

Results

To ensure that FTHL17 was strictly testis-specific, FTHL17 expression in normal tissues was analyzed by RT-PCR (Table 9). No signal was detected in any normal tissue tested, other than testis.

RT-PCR was used to screen a large number of tumoral samples of various histological origins (Table 10). Overall, expression of FTHL17 appeared to be reactivated in 6% of all tested tumors. It was expressed in 27% (3/11) lung carcinomas, and in 22% (2/9) bladder carcinomas.

RT-real-time-PCR was used to quantify the FTHL17 expression in some tumoral samples. Table 11 shows that FTHL17 was expressed at high levels in some tumors (up to 283% of the testis expression level).

TABLE 9
FTHL17 expression in normal tissues

| Tissue sample | Patient code | RT-PCR analysis |
|---|---|---|
| Testis | LB 882 | ++ |
| Testis | CLO 26 | ++ |
| Adrenal gland | CLO 32 | − |
| Bone marrow | CLO 21 | − |
| Brain | CLO 14 | − |
| Colon | CLO 20 | − |
| Heart | CLO 15 | − |
| Kidney | CLO 16 | − |
| Liver | CLO 17 | − |
| Lung | LB 195 | − |
| Lung | CLO 18 | − |
| Melanocytes | LB 1878 | − |
| Muscle | CLO 25 | − |
| Ovary | LB 2040 | − |
| Placenta | CLO 31 | − |
| Prostate | CLO 24 | − |
| Spleen | CLO 22 | − |
| Thymus | CLO 23 | − |
| Trachea | CLO 19 | − |
| Uterus | CLO 27 | − |

++ and − refer to the PCR signal intensity

TABLE 10
RT-PCR analysis of FTHL 17 expression in tumor samples

| | Number of positive tumoral samples/ | |
|---|---|---|
| Tumoral samples | Number of tumoral samples tested | |
| Lung carcinomas NSCLC | 3/11 | (27%) |
| Bladder carcinomas | 2/9 | (22%) |
| Breast carcinomas | 1/7 | |
| Head and neck | 1/10 | |
| epidermoid carcinomas | | |
| Renal tumors | 0/9 | |
| Sarcomas | 0/8 | |
| Prostate tumors | 1/6 | |
| Uterine tumors | 0/5 | |
| Thyroid tumors | 0/4 | |
| Brain tumors | 0/4 | |
| Leukemias | 0/4 | |
| Esophagial carcinomas | 0/5 | |
| Myelomas | 0/3 | |
| Neuroblastomas | 0/2 | |
| Cutaneous melanomas | 0/13 | |
| Colorectal carcinomas | 0/11 | |
| Mcsotheliomas | 0/1 | |
| | 8/112 | (7%) |

TABLE 11
Quantification of FTHL17 level expression in tumoral sample

| | | RT-real-time-PCR analysis |
|---|---|---|
| | | \overline{Percent of NXF2 level expression in} |
| Tissue sample | Patient code | tumoral tissues referred to testis. |
| Normal testis | HM 31 1A5 | 100 |
| Bladder tumor | HM 50 | 83 |
| Bladder tumor | HM 87 | 34 |
| Breast tumor | LB 208 | 135 |
| Head and neck | LB 1014 | 106 |
| carcinoma | | |
| Lung carcinoma | LB 1136 | 283 |
| NSCLC | | |
| Lung carcinoma | LB 1135 | 240 |
| NSCLC | | |
| Lung carcinoma | LB 1061 | 3 |
| NSCLC | | |
| Prostate tumor | LB 541 | 7 |

### Example 4

### TEX15 Testis-Expressed Gene 15

Introduction

TEX15 is located on the chromosome 8. Its function is unknown. TEX15 is listed in GenBank under Accession umber: AF285605. The expression of TEX15 was tested in 17 tumoral cell lines by RT-PCR. TEX15 expression was detected in ten cell lines, including five melanomas cell lines, four lung cell lines and one sarcoma cell line.

Methods

RT-PCR Conditions

Reverse transcription was performed on 2 μg of total RNA with 200 units of M-MLV reverse transcriptase (GibcoBRL-Life Technologies). cDNA corresponding to 50 ng of reverse-transcribed RNA was then amplified by PCR with primers TEX15_sens 5′-gtctgcatgtaacccaacata-3′ (SEQ ID NO: 20) and TEX15_as 5′-gtgtcctggatgaaaggatt-3′ (SEQ ID NO: 21), and with 0.625 units of TaKaRa Taq polymerase. Cycling conditions were 30 cycles (30 sec at 94° C.; 30 sec at 60° C.; 1 min at 72° C.) and 10 min at 72° C. for final extension. No product was amplified from human genomic DNA under these conditions. Specific amplification was confirmed by the sequencing of the testis PCR product.

RT-Real-Time PCR Conditions

The quality and the concentration of RNA samples were checked in an ethidium bromide staining gel. The relative amounts of RNA were corrected according to the intensity of the RNA 28S band measured with a Kodak Digital Science Image Station 440CF (Eastman Kodak Co.). 0.75 μg of total RNA samples were reverse transcribed with 200 units of M-MLV reverse transcriptase (GibcoBRL-Life Technologies). The quantitation of TEX15 mRNA levels was carried out by a real-time fluorescence detection method, using the ABI Prism 7700 sequence detector (Applied Biosystems). cDNA corresponding to 1.875 ng of reverse transcribed RNA was amplified by real-time-PCR with 0.625 units Hot GoldStar enzyme and 0.25 units Uracyl-N-glycosylase (Eurogentec SA). The reaction was performed using the TEX 15 primers described above and 5′FAM-tcagtacagcaacagcaatggca-3′TAMRA (SEQ ID NO: 22) as a probe. The thermal conditions were 2 min at 50° C., 10 min at 95° C., 50 cycles (15 sec at 95° C.; 2 min at 60° C.).

Results

To ensure that TEX15 was strictly testis-specific, TEX15 expression in normal tissues was analyzed by RT-PCR (Table 12.). No signal was detected in almost all normal tissue tested except in brain, ovary, and uterus where a very weak signal was detected. The level of TEX 15 expression in these tissues was quantified by RT-real-time PCR and found to represent only 0.5 to 1.8% of the expression level in testis.

RT-PCR was used to screen a large number of tumoral samples of various histological origins. (Table 13). Overall, expression of TEX15 appeared to be reactivated in 14% of all tested tumors. Its expression frequency reached 28% (4/14) in bladder carcinomas, 28% (2/7) in sarcomas, 27% (3/11) in cutaneous melanomas, 21% (4/19) in lung carcinomas and 20% (2/10) in esophageal carcinomas.

RT-real-time-PCR was used to quantify the TEX 15 expression in some tumoral samples. Table 14 shows that TEX15 was expressed at high levels in some tumors (up to 90% of the testis expression level).

TABLE 12
TEX15 expression in normal samples

| | | | RT-real-time-PCR analysis |
|---|---|---|---|
| | | | Percent of TEX15 level |
| | | RT-PCR | expression in_normal |
| Tissue sample | Patient code | analysis | tissues in reference to testis |
| Testis | LB 882 | +++ | / |
| Testis | CLO 26 | +++ | 100% |
| Adrenal gland | CLO 32 | − | / |
| Bone marrow | CLO 21 | − | / |
| Brain | 878 JNO 9 | − | / |
| Brain | CLO 14 | + | 1.5% |
| Colon | CLO 20 | − | / |
| Heart | CLO 15 | − | / |
| Kidney | CLO 16 | − | / |
| Liver | CLO 17 | − | / |
| Lung | CLO18 | − | / |
| Muscle | CLO 25 | − | / |
| Ovary | LB 2040 | + | 1.8% |
| Ovary | CLO 7 64036 | − | / |
| Placenta | CLO 31 | − | / |
| Prostate | CLO 24 | − | / |
| Skin | LB 243 | − | / |
| Spleen | CLO 22 | − | / |
| Thymus | CLO 23 | − | / |
| Trachea | CLO 19 | − | / |
| Uterus | LB 1031 | − | / |
| Uterus | CLO 27 | + | 0.5% |

+++, + and − refer to the PCR signal intensity
/: not tested.

TABLE 13
RT-PCR analysis of TEX 15 expression in tumor samples

| | Number of positive tumoral samples/ | |
|---|---|---|
| Tumoral samples | Number of tumoral samples tested | |
| Bladder carcinomas | 4/14 | (28%) |
| Sarcomas | 2/7 | (28%) |
| Cutaneous melanomas | 3/11 | (27%) |
| Lung carcinomas NSCLC | 4/19 | (21%) |
| Esophagial carcinomas | 2/10 | (20%) |
| Head and neck epidermoid | 1/9 | |
| carcinoma | | |
| Prostate tumors | 1/8 | |
| Renal tumors | 1/3 | |
| Myelomas | 0/10 | |
| Neuroblastomas | 1/1 | |
| Breast carcinomas | 0/8 | |
| Colorectal carcinomas | 0/8 | |
| Thyroid tumors | 0/1 | |
| Uterine tumors | 0/3 | |
| Leukemias | 0/22 | |
| Total | 19/134 | (14%) |

TABLE 14
Quantification of TEX15 level expression in tumor samples

| | | RT-real-time-PCR analysis |
|---|---|---|
| | | \overline{Percent of NXF2 level expression in} |
| Tissue sample | Patient code | tumoral tissues referred to testis. |
| Normal testis | HM 31 1A5 | 100 |
| Bladder tumor | HM 50 | 2 |
| Cutaneous | CPVB 50.II | 90 |
| melanoma | | |
| Cutaneous | CPVB 50.I | 7 |
| melanoma | | |
| Cutaneous | EN 7 | 7 |
| melanoma | | |
| Esophagial tumor | LB 1472 | 24 |
| Esophagial tumor | LB 1208 | 13 |
| Head and neck | BB 61 | 12 |
| carcinoma | | |
| Lung carcinoma | LB 1123 | 26 |
| NSCLC | | |
| Lung carcinoma | LB 1061 | 11 |
| NSCLC | | |
| Lung carcinoma | LB 1152 | 9 |
| NSCLC | | |
| Lung carcinoma | LB 1135 | 6 |
| NSCLC | | |
| Neuroblastoma | LB 837 | 24 |
| Prostate tumor | LB 1406 | 13 |
| Renal tumor | LB 311 | 16 |
| Sarcoma | LB 1443 | 24 |

### Example 5

### TDRD1 Tudor Domain Protein 1

Introduction

TDRD1 is located on the chromosome 10. Its function is unknown. TDRD1 is listed in GenBank under Accession number AF285606. The expression of TDRD1 was tested in 17 tumoral cell lines by RT-PCR. TDRD1 expression was detected in 3 cell lines, including two melanoma cell line and one lung carcinoma cell line.

Methods

RT-PCR Conditions

Reverse transcription was performed on 2 μg of total RNA with 200 units of M-MLV reverse transcriptase (GibcoBRL-Life Technologies). cDNA corresponding to 50 ng of reverse-transcribed RNA was then amplified by PCR with primers TDRD1_sens 5′-ggtagttggtatcgtgcttta-3′ (SEQ ID NO: 23) and TDRD1_as 5′-gaacatcactaattattctggga-3′ (SEQ ID NO: 24), and with 0.625 units of TaKaRa Taq polymerase. Cycling conditions were 30 cycles (30 sec at 94° C.; 30 sec at 60° C.; 1 min at 72° C.) and 10 min at 72° C. for final extension. As the primers where chosen in different exons, PCR products originating from RNA could easily be distinguished from those produced by contaminating genomic DNA. Specific amplification was confirmed by the sequencing of the testis PCR product.

RT-Real-Time PCR Conditions

The quality and the concentration RNA samples were checked in an ethidium bromide staining gel. The relative amounts of RNA were corrected according to the intensity of the RNA 28S band measured with a Kodak Digital Science Image Station 440CF (Eastman Kodak Co.). 0.75 μg of total RNA samples were reverse transcribed with 200 units of M-MLV reverse transcriptase (GibcoBRL-Life Technologies). The quantitation of TDRD1 mRNA levels was carried out by a real-time fluorescence detection method, using the ABI Prism 7700 sequence detector (Applied Biosystems). cDNA corresponding to 1.875 ng of reverse transcribed RNA was amplified by real-time-PCR with 0.625 units Hot GoldStar enzyme and 0.25 units Uracyl-N-glycosylase (Eurogentec SA). The reaction was performed using the TDRD1 primers described above and 5′FAM-cctttcagggaatacggtgcc-3′TAMRA (SEQ ID NO: 25) as a probe. The thermal conditions were 2 min at 50° C., 10 min at 95° C., 50 cycles (15 sec at 95° C.; 1 min at 60° C.).

Results

To ensure that TDRD1 was strictly testis-specific, TDRD1 expression in normal tissues was analyzed by RT-PCR (Table 15). No signal was detected in almost all normal tissue tested except in brain, ovary, thymus, spleen, colon, and trachea, where a very weak signal was detected. The level of TDRD1 expression in these tissues was quantified by RT-real-time PCR and found to represent only 0.2 to 1.5% of the expression level in testis.

RT-PCR was used to screen a large number of tumoral samples of various histological origins (Table 16). Overall, expression of TDRD1 appeared to be reactivated in 13% of all tested tumors. Its expression frequency reached 57% (4/7) in prostate tumors, 50% (4/8) in breast tumors, 28% (4/14) in bladder carcinomas, and 22% (2/9) in sarcomas.

RT-real-time-PCR was used to quantify the TDRD1 expression in some tumoral samples. Table 17 shows that TDRD1 was expressed at very high levels in some tumors (up to 821% of the testis expression level).

TABLE 15
TDRD1 expression in normal samples

| | | | RT-real-time-PCR analysis |
|---|---|---|---|
| | | | Percent of TDRD1 level |
| | | RT-PCR | expression in normal |
| Tissue sample | Patient code | analysis | tissues in reference to testis |
| Testis | LB 882 | +++ | / |
| Testis | CLO 26 | +++ | 100% |
| Adrenal gland | CLO 32 | − | / |
| Bone marrow | CLO 21 | − | / |
| Brain | 878 JNO 9 | − | / |
| Brain | CLO 14 | + | 0.3% |
| Colon | CLO 20 | + | 0.9% |
| Heart | CLO 15 | − | / |
| Kidney | CLO 16 | − | / |
| Liver | CLO 17 | − | / |
| Lung | CLO 18 | − | / |
| Muscle | CLO 25 | − | / |
| Ovary | LB 2040 | + | 0.3% |
| Ovary | CLO 7 64036 | +/− | / |
| Placenta | CLO 31 | − | / |
| Prostate | CLO 24 | − | / |
| Skin | LB 243 | − | / |
| Spleen | CLO 22 | +/− | 0.2% |
| Thymus | CLO 23 | +/− | 0.3% |
| Trachea | CLO 19 | + | 1.5% |
| Uterus | LB 1031 | − | / |
| Uterus | CLO 27 | − | / |

+++, +/− and − refer to the PCR signal intensity
/: not tested.

TABLE 16
RT-PCR analysis of TDRD1 expression in tumor samples

| | Number of positive tumoral samples/ | |
|---|---|---|
| Tumoral samples | Number of tumoral samples tested | |
| Prostate tumors | 4/7 | (57%) |
| Breast carcinomas | 4/8 | (50%) |
| Bladder carcinomas | 4/14 | (28%) |
| Head and neck epidermoid | 2/9 | (22%) |
| carcinoma | | |
| Leukemias | 1/20 | |
| Lung carcinoma NSCLC | 1/19 | |
| Esophagial carcinomas | 1/10 | |
| Sarcomas | 0/7 | |
| Colorectal carcinomas | 0/8 | |
| Renal tumors | 0/3 | |
| Uterine tumors | 0/3 | |
| Neuroblastomas | 0/1 | |
| Thyroid tumors | 0/1 | |
| Cutaneous melanomas | 0/11 | |
| Myelomas | 0/10 | |
| Total | 17/131 | (13%) |

TABLE 17
Quantification of TDRD1 level expression in tumoral samples

| | | RT-real-time-PCR analysis |
|---|---|---|
| | | \overline{Percent of NXF2 level expression in} |
| Tissue sample | Patient code | tumoral tissues referred to testis. |
| Normal testis | HM 31 1A5 | 100 |
| Bladder carcinoma | HM 50 | 7 |
| Breast tumor | LB 234 | 50 |
| Breast tumor | LB 208 | 39 |
| Breast tumor | LB 235 | 34 |
| Esophagial tumor | LB 1472 | 7 |
| Head and neck | BB 67 | 274 |
| carcinoma | | |
| Head and neck | BB 50 | 24 |
| carcinoma | | |
| Leukemia | LB 1021 | 821 |
| Lung carcinoma | LB 264 | 25 |
| Prostate tumor | LB 1406 | 653 |
| Prostate tumor | LB 295 | 43 |
| Prostate tumor | LB 505 | 22 |
| Prostate tumor | JH 24 | 19 |

Other aspects of the invention will be clear to the skilled artisan and need not be repeated here. Each reference cited herein is incorporated by reference in its entirety.

The terms and expressions that have been employed are used as terms of description and not of limitation, and there is no intention in the use of such terms and expressions of excluding any equivalents of the features shown and described or portions thereof, it being recognized that various modifications are possible within the scope of the invention.

1. A method for diagnosing cancer in a subject comprising:
obtaining a non-testis biological sample from a subject,
determining the expression in the sample of one or more cancer-associated nucleic acid molecules comprising a nucleotide sequence selected from the group consisting of SEQ ID NO: 1-5,
wherein if the nucleotide sequence is SEQ ID NO: 1, the biological sample is not a tracheal sample,
wherein if the nucleotide sequence is SEQ ID NO: 4, the biological sample is not a brain, ovarian, or uterine sample,
wherein if the nucleotide sequence is SEQ ID NO: 5, the biological sample is not a brain, ovarian, thymus, spleen, colon, or tracheal sample, and
wherein expression of the nucleic acid molecule in the sample is diagnostic for cancer in the subject.

2. The method of claim 1, wherein expression is determined for at least 2, 3, 4, or 5 nucleic acid molecules comprising a nucleotide sequence selected from the group consisting of SEQ ID NOs:1-5.

3. (canceled)

4. The method of claim 1, wherein the cancer is selected from the group consisting of: cutaneous melanomas, sarcomas, lung carcinomas, bladder carcinomas, epidermoid carcinoma, renal tumors, breast carcinomas, prostate tumors, esophageal carcinomas, leukemias, uterine tumors, brain tumors, thyroid tumors, neuroblastomas, colorectal carcinomas, mesotheliomas, uveal melanomas, and myelomas.

5-8. (canceled)

9. A method for diagnosing cancer in a subject comprising:
obtaining a non-testis biological sample from a subject,
determining the level of expression of a cancer-associated nucleic acid molecule comprising one or more nucleotide sequences selected from the group consisting of SEQ ID NOs: 1-5,
comparing the level of expression of the nucleic acid molecule in the subject sample to a level of expression of the nucleic acid in a testis control tissue, wherein a determination that the level of expression of the nucleic acid in the sample from the subject is greater than about 1.8% of the level of expression of the nucleic acid in the control tissue, is diagnostic for cancer in the subject.

10. The method of claim 9, wherein the level of expression of the nucleic acid in the sample from the subject is at least about 1.9%, 2.0%, 2.5%, 3.0%, 4%, 5%, 10%, 20%, 30%, 40%, 50%, 60%, 70%, 80%, or 90% of the level of expression in the control tissue.

11. The method of claim 9, wherein expression is determined for at least 2, 3, 4, or 5 nucleic acid molecules comprising a nucleotide sequence selected from the group consisting of SEQ ID NOs:1-5.

12. (canceled)

13. The method of claim 9, wherein the cancer is selected from the group consisting of: cutaneous melanomas, sarcomas, lung carcinomas, bladder carcinomas, epidermoid carcinoma, renal tumors, breast carcinomas, prostate tumors, esophageal carcinomas, leukemias, uterine tumors, brain tumors, thyroid tumors, neuroblastomas, colorectal carcinomas, mesotheliomas, uveal melanomas, and myelomas.

14-17. (canceled)

18. A method for determining onset, progression, or regression, of cancer in a subject comprising:
obtaining from a subject two non-testis biological samples, wherein the samples comprise the same tissue type and are obtained at different times,
determining a level of expression of one or more cancer-associated nucleic acid molecules or expression products thereof in the two samples, wherein the nucleic acid molecules comprise nucleotide sequences selected from the group consisting of: SEQ ID NOs: 1-5,
wherein if the nucleotide sequence is SEQ ID NO: 1, the biological sample is not a tracheal sample,
wherein if the nucleotide sequence is SEQ ID NO: 4, the biological sample is not a brain, ovarian, or uterine sample,
wherein if the nucleotide sequence is SEQ ID NO: 5, the biological sample is not a brain, ovarian, thymus, spleen, colon, or tracheal sample, and
comparing the levels of expression in the two samples
wherein a higher level of expression of the one or more cancer-associated nucleic acid molecules in the first non-testis sample than in the second non-testis sample indicates regression of cancer,
wherein a lower level of expression of the one or more cancer-associated nucleic acid molecules in the first non-testis sample than the second non-testis sample indicates progression of cancer, and
wherein a level of expression of the one or more cancer-associated nucleic acid molecules in the first non-testis sample that is less than the level of expression of the cancer-associated nucleic acid molecules in the second testis sample indicates onset of cancer.

19. The method of claim 18, wherein expression is determined for at least 2, 3, 4, or 5 nucleic acid molecules comprising a nucleotide sequence selected from the group consisting of SEQ ID NOs:1-5.

20. The method of claim 18, wherein the expression products are polypeptides comprising amino acid sequences selected from the group consisting of SEQ ID NOs: 6-10.

21-24. (canceled)

25. The method of claim 18, wherein the cancer is selected from the group consisting of: cutaneous melanomas, sarcomas, lung carcinomas, bladder carcinomas, epidermoid carcinoma, renal tumors, breast carcinomas, prostate tumors, esophageal carcinomas, leukemias, uterine tumors, brain tumors, thyroid tumors, neuroblastomas, colorectal carcinomas, mesotheliomas, uveal melanomas, and myelomas.

26-28. (canceled)

29. A method for determining onset, progression, or regression, of cancer in a subject comprising:
obtaining from a subject a first and a second non-testis biological sample, wherein the samples comprise the same tissue type and are obtained at different times,
determining a level of expression of one or more cancer-associated nucleic acid molecules or expression products thereof in the first and second non-testis biological samples, wherein the nucleic acid molecules comprise nucleotide sequences selected from the group consisting of: SEQ ID NOs: 1-5,
comparing the level of expression of one or more cancer-associated nucleic acid molecules of the first and the second non-testis biological samples to the level of expression of the one or more cancer-associated nucleic acid molecules in a testis control sample,
wherein a determination that the level of the one or more cancer-associated nucleic acid molecules in either the first or second non-testis biological samples is more than 1.8% of the level of expression of the one or more cancer-associated nucleic acid molecules in the testis control sample, is an diagnostic for cancer,
wherein a higher level of expression of the one or more cancer-associated nucleic acid molecules in the first non-testis sample than in the second non-testis sample indicates regression of cancer,
wherein a lower level of expression of the one or more cancer-associated nucleic acid molecules in the first non-testis sample than the second non-testis sample indicates progression of cancer, and
wherein a level of expression of the one or more cancer-associated nucleic acid molecules in the first non-testis sample that is less than the level of expression of the one or more cancer-associated nucleic acid molecules in the control testis sample and the level of expression of the cancer-associated nucleic acid molecules in the second testis sample indicates onset of cancer.

30. The method of claim 29, wherein the level of expression of the one or more nucleic acid molecules in the sample from the subject is at least about 1.9%, 2.0%, 2.5%, 3.0%, 4%, 5%, 10%, 20%, 30%, 40%, 50%, 60%, 70%, 80%, or 90% of the level of expression in the control tissue.

31. The method of claim 29, wherein expression is determined for at least 2, 3, 4, or 5 nucleic acid molecules comprising a nucleotide sequence selected from the group consisting of SEQ ID NOs:1-5.

32. (canceled)

33. The method of claim 29, wherein the cancer is selected from the group consisting of: cutaneous melanomas, sarcomas, lung carcinomas, bladder carcinomas, epidermoid carcinoma, renal tumors, breast carcinomas, prostate tumors, esophageal carcinomas, leukemias, uterine tumors, brain tumors, thyroid tumors, neuroblastomas, colorectal carcinomas, mesotheliomas, uveal melanomas, and myelomas.

34-37. (canceled)

38. A method for diagnosing cancer in a subject comprising:
obtaining a non-testis biological sample from a subject,
contacting the sample with antibodies or antigen-binding fragments thereof, that bind specifically to one or more different cancer-associated polypeptides encoded by nucleic acid molecules comprising a nucleotide sequence selected from the group consisting of SEQ ID NOs: 1-5,
wherein if the nucleotide sequence is SEQ ID NO: 1, the biological sample is not a tracheal sample,
wherein if the nucleotide sequence is SEQ ID NO: 4, the biological sample is not a brain, ovarian, or uterine sample,
wherein if the nucleotide sequence is SEQ ID NO: 5, the biological sample is not a brain, ovarian, thymus, spleen, colon, or tracheal sample, and
wherein specific binding of one or more antibodies or antigen-binding fragments thereof in the sample is diagnostic for cancer in the subject.

39. The method of claim 38, wherein specific binding is determined for at least 2, 3, 4, or 5 polypeptides encoded by nucleic acid molecules comprising a nucleotide sequence selected from the group consisting of SEQ ID NOs:1-5.

40. (canceled)

41. The method of claim 38, wherein the cancer is selected from the group consisting of: cutaneous melanomas, sarcomas, lung carcinomas, bladder carcinomas, epidermoid carcinoma, renal tumors, breast carcinomas, prostate tumors, esophageal carcinomas, leukemias, uterine tumors, brain tumors, thyroid tumors, neuroblastomas, colorectal carcinomas, mesotheliomas, uveal melanomas, and myelomas.

42-43. (canceled)

44. A method for diagnosing cancer in a subject comprising:
obtaining a non-testis biological sample from a subject,
contacting the sample with antibodies or antigen-binding fragments thereof, that bind specifically to one or more different cancer-associated polypeptides encoded by nucleic acid molecules comprising a nucleotide sequence selected from the group consisting of SEQ ID NOs:1-5,
determining the level of specific binding between the antibodies or antigen-binding fragments thereof and cancer-associated polypeptides in the sample,
comparing the level of specific binding between the antibodies or antigen-binding fragments thereof and cancer-associated polypeptides in a testis control tissue, wherein a determination that the level of specific binding in the sample from the subject is more than 1.8% of the level of specific binding in the control tissue, is diagnostic for cancer in the subject.

45. The method of claim 44, wherein expression is determined for at least 2, 3, 4, or 5 polypeptides encoded by nucleic acid molecules comprising a nucleotide sequence selected from the group consisting of SEQ ID NOs:1-5.

46. (canceled)

47. The method of claim 44, wherein the cancer is selected from the group consisting of: cutaneous melanomas, sarcomas, lung carcinomas, bladder carcinomas, epidermoid carcinoma, renal tumors, breast carcinomas, prostate tumors, esophageal carcinomas, leukemias, uterine tumors, brain tumors, thyroid tumors, neuroblastomas, colorectal carcinomas, mesotheliomas, uveal melanomas, and myelomas.

48-50. (canceled)

51. A method for determining onset, progression, or regression, of cancer in a subject comprising:
obtaining from a subject two non-testis biological samples, wherein the samples comprise the same tissue type and are obtained at different times,
contacting the two samples with antibodies or antigen-binding fragments thereof, that bind specifically to one or more cancer-associated polypeptides encoded by nucleic acid molecules comprising a nucleotide sequence selected from the group consisting of SEQ ID NO:s: 1-5,
determining a level of specific binding between the one or more cancer-associated polypeptides in the at least two samples,
wherein if the nucleotide sequence is SEQ ID NO: 1, the biological sample is not a tracheal sample,
wherein if the nucleotide sequence is SEQ ID NO: 4, the biological sample is not a brain, ovarian, or uterine sample,
wherein if the nucleotide sequence is SEQ ID NO: 5, the biological sample is not a brain, ovarian, thymus, spleen, colon, or tracheal sample, and
comparing the levels of specific binding of the one or more cancer-associated polypeptides in the at least two samples to determine the onset, progression, or regression of the cancer,
wherein a higher level of specific binding of the one or more cancer-associated polypeptides in the first sample than in the second sample indicates regression of cancer,
wherein a lower level of specific binding of the one or more cancer-associated polypeptides in the first sample than the second non-testis sample indicates progression of cancer, and
wherein a level of specific binding of the one or more cancer-associated polypeptides in the first non-testis sample that is less than the level of expression of the cancer-associated nucleic acid in the second testis sample indicates onset of cancer.

52. (canceled)

53. The method of claim 51, wherein the cancer is selected from the group consisting of: cutaneous melanomas, sarcomas, lung carcinomas, bladder carcinomas, epidermoid carcinoma, renal tumors, breast carcinomas, prostate tumors, esophageal carcinomas, leukemias, uterine tumors, brain tumors, thyroid tumors, neuroblastomas, colorectal carcinomas, mesotheliomas, uveal melanomas, and myelomas.

54-59. (canceled)

60. A method for determining onset, progression, or regression, of cancer in a subject comprising:
obtaining from a subject two non-testis biological samples, wherein the samples comprise the same tissue type and are obtained at different times,
contacting the two samples with antibodies or antigen-binding fragments thereof, that bind specifically to one or more cancer-associated polypeptides encoded by nucleic acid molecules comprising a nucleotide sequence selected from the group consisting of SEQ ID NO:s: 1-5,
determining a level of specific binding between the one or more cancer-associated polypeptides in the at least two samples,
comparing the levels of specific binding of the one or more cancer-associated polypeptides in the two samples to the level of specific binding of the one or more cancer-associated polypeptides in a testis control sample,
wherein a determination that the level of specific binding of the one or more cancer-associated polypeptides in either the first or second non-testis sample is more than 1.8% of the level of specific binding of the one or more cancer associated polypeptides in the testis control sample, is diagnostic for cancer,
wherein a higher level of specific binding of the one or more cancer-associated polypeptides in the first non-testis sample than the second non-testis sample indicates regression of cancer,
wherein a lower level of specific binding of the one or more cancer-associated polypeptides in the first non-testis sample than the second non-testis sample indicates progression of cancer, and
wherein a level of specific binding of the one or more cancer-associated polypeptides in the first non-testis sample that is less than the level of specific binding of the one or more cancer-associated polypeptides in a control testis sample and is less than the level of binding of the one or more cancer-associated polypeptides in the second testis sample indicates onset of cancer.

61. (canceled)

62. The method of claim 60, wherein the cancer is selected from the group consisting of: cutaneous melanomas, sarcomas, lung carcinomas, bladder carcinomas, epidermoid carcinoma, renal tumors, breast carcinomas, prostate tumors, esophageal carcinomas, leukemias, uterine tumors, brain tumors, thyroid tumors, neuroblastomas, colorectal carcinomas, mesotheliomas, uveal melanomas, and myelomas.

63-70. (canceled)

71. A method for treating a subject with cancer characterized by increased expression or activity of a cancer-associated polypeptide, comprising:
administering to the subject an effective amount of an antibody or an antigen-binding fragment thereof to a cancer-associated polypeptide comprising an amino acid sequence selected from the group consisting of SEQ ID NOs:6-10 to treat the cancer.

72. The method of claim 71, wherein the antibodies or antigen-binding fragments are labeled with one or more cytotoxic agents.

73-76. (canceled)

77. The method of claim 71, wherein the cancer is selected from the group consisting of: cutaneous melanomas, sarcomas, lung carcinomas, bladder carcinomas, epidermoid carcinoma, renal tumors, breast carcinomas, prostate tumors, esophageal carcinomas, leukemias, uterine tumors, brain tumors, thyroid tumors, neuroblastomas, colorectal carcinomas, mesotheliomas, uveal melanomas, and myelomas.

78. A method for selecting a course of treatment of a subject having or suspected of having cancer, comprising:
obtaining from the subject a biological sample,
contacting the sample with antibodies or antigen-binding fragments thereof that bind specifically to one or more different cancer-associated polypeptides encoded by nucleic acid molecules comprising a nucleotide sequence selected from the group consisting of SEQ ID NOs:1-5,
wherein if the nucleotide sequence is SEQ ID NO: 1, the biological sample is not a tracheal sample,
wherein if the nucleotide sequence is SEQ ID NO: 4, the biological sample is not a brain, ovarian, or uterine sample,
wherein if the nucleotide sequence is SEQ ID NO: 5, the biological sample is not a brain, ovarian, thymus, spleen, colon, or tracheal sample,
determining specific binding between cancer-associated polypeptides in the sample that are differentially expressed in different types of cancer, and the antibodies or antigen-binding fragments thereof, and
selecting a course of treatment appropriate to the cancer of the subject.

79. (canceled)

80. The method of claim 78, wherein the treatment is administering antibodies that specifically bind to the cancer-associated polypeptides.

81. The method of claim 80, wherein the antibodies are labeled with one or more cytotoxic agents.

82. (canceled)

83. The method of claim 78, wherein the cancer is selected from the group consisting of: cutaneous melanomas, sarcomas, lung carcinomas, bladder carcinomas, epidermoid carcinoma, renal tumors, breast carcinomas, prostate tumors, esophageal carcinomas, leukemias, uterine tumors, brain tumors, thyroid tumors, neuroblastomas, colorectal carcinomas, mesotheliomas, uveal melanomas, and myelomas.

84-93. (canceled)

94. A method for diagnosing cancer in a subject comprising:
contacting a non-testis tissue in a subject with an antibody that selectively binds to a cancer-associated polypeptide comprising an amino acid sequence selected from the group consisting of SEQ ID NOs:6-10,
determining the binding in the tissue of the antibody, wherein binding of the antibody in the tissue is diagnostic for cancer in the subject.

95. (canceled)

96. The method of claim 94, wherein the cancer is selected from the group consisting of: cutaneous melanomas, sarcomas, lung carcinomas, bladder carcinomas, epidermoid carcinoma, renal tumors, breast carcinomas, prostate tumors, esophageal carcinomas, leukemias, uterine tumors, brain tumors, thyroid tumors, neuroblastomas, colorectal carcinomas, mesotheliomas, uveal melanomas, and myelomas.

97-98. (canceled)
